SEQUENCE LISTING (25 sequences)

SEQ ID NO: 1 - DNA, 4673 bp, synthetic construct
  misc_feature 1..4673: note = KTXHIS
  source 1..4673: mol_type = other DNA
atctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccaataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcatcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcttgtcagtaacgagaaggtcgcgaatccaggcgctttttagactggtcgtaatgaaattcaggaggaattccatatggcggccgcactcgagcaccaccaccaccaccactgatgaagacggccgcctaggggtaccgagctcggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagactaggcttccgcgccctcatccgaaagggcgtattcatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtct

SEQ ID NO: 2 - DNA, 180 bp, synthetic construct
  misc_feature 1..180: note = Multiple cloning site - MCS
  source 1..180: mol_type = other DNA
gagaaggtcgcgaatccaggcgctttttagactggtcgtaatgaaattcaggaggaattccatatggcggccgcactcgagcaccaccaccaccaccactgatgaagacggccgcctaggggtaccgagctcggatcctctagagtcgacctgcaggcatgcaagcttggctgttttggc

SEQ ID NO: 3 - protein, 214 aa, synthetic construct
  REGION 1..214: note = Certolizumab light chain
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 229 aa, synthetic construct
  REGION 1..229: note = Certolizumab heavy chain
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 5 - DNA, 645 bp, synthetic construct
  misc_feature 1..645: note = Certolizumab light chain - DNA
  source 1..645: mol_type = other DNA
gatattcagatgactcagagcccaagttcgctgagcgcttctgttggcgatcgtgtgaccattacatgcaaagcctcacagaacgttggtaccaatgtcgcctggtatcagcagaaacctggaaaagcgcccaaagcgctcatctactcagcgagcttcctgtattcaggcgtgccgtatcgctttagcggctctggttccggtacagactttaccctcacgatttcgtccttacaaccggaagatttcgccacgtactattgccagcaatacaacatctatccgctgacctttggacaaggcaccaaagtggagatcaaacgcactgttgctgcaccgagtgtgttcatctttccaccgtctgatgagcagctgaagtctggtacagcaagtgttgtgtgtctgctgaacaacttctatccgcgtgaagctaaagtacagtggaaagtcgacaatgccttgcaatccgggaatagccaggaaagcgtgactgaacaggacagcaaggattcgacctacagtctgagcagtaccttaaccttgtcgaaagcggattacgagaaacacaaggtctatgcctgtgaagtcacgcatcaaggcctgtcatcgcctgttactaaatcatttaatagaggagaatgttaa

SEQ ID NO: 6 - DNA, 690 bp, synthetic construct
  misc_feature 1..690: note = Certolizumab heavy chain - DNA
  source 1..690: mol_type = other DNA
gaagtgcagcttgtggagtctggaggtggcttagtccagccaggtggttccctgcgcttgtcctgtgcagcgagcgggtatgtattcacagattatggcatgaactgggttcggcaagcaccaggcaaaggcctcgaatggatggggtggatcaacacgtatattggggaaccgatttatgcggatagcgtcaaaggtcgcttcacgttcagtctggataccagcaaatcaaccgcgtatctccagatgaatagcctccgtgctgaagatactgccgtgtactactgtgcgcgtggttatcgcagttatgcgatggattactggggccaaggcaccttagtcaccgttagttctgcctccaccaaaggcccatcagtgtttccgctggccccttcgtctaaatcgacgagtggtggcacagccgcactgggatgcctggtcaaagactactttcccgaacctgtaaccgtaagctggaatagtggtgctttgacctcaggcgtgcatacgtttccggctgtcctgcagtcatccggtctgtactcgctttcgagcgttgttactgtaccctctagctccctgggcacccagacgtacatctgcaatgtgaaccataagccgtcgaacaccaaagtggacaagaaagttgagccgaaaagctgcgacaaaacgcacacatgtgccgcctaa

SEQ ID NO: 7 - protein, 22 aa, Pectobacterium carotovorum
  REGION 1..22: note = PelB signal peptide - aa
MKYLLPTAAAGLLLLAAQPAMA

SEQ ID NO: 8 - DNA, 119 bp, synthetic construct
  misc_feature 1..119: note = rhaBAD promoter
  source 1..119: mol_type = other DNA
caccacaattcagcaaattgtgaacatcatcacgttcatctttccctggttgccaatggcccattttcttgtcagtaacgagaaggtcgcgaatccaggcgctttttagactggtcgta

SEQ ID NO: 9 - DNA, 936 bp, synthetic construct
  misc_feature 1..936: note = RhaR transcription activator
  source 1..936: mol_type = other DNA
atggctttctgcaataacgcgaatcttctcaacgtatttgtacgccatattgcgaataatcaacttcgttctctggccgaggtagccacggtggcgcatcagttaaaacttctcaaagatgatttttttgccagcgaccagcaggcagtcgctgtggctgaccgttatccgcaagatgtctttgctgaacatacacatgatttttgtgagctggtgattgtctggcgcggtaatggcctgcatgtactcaacgatcgcccttatcgcattacccgtggcgatctcttttacattcatgctgatgataaacactcctacgcttccgttaacgatctggttttgcagaatattatttattgcccggagcgtctgaagctgaatcttgactggcagggggcgattccgggatttaacgccagcgcagggcaaccacactggcgcttaggtagcatggggatggcgcaggcgcggcaggttattggtcagcttgagcatgaaagtagtcagcatgtgccgtttgctaacgaaatggctgagttgctgttcgggcagttggtgatgttgctgaatcgccatcgttacaccagtgattcgttgccgccaacatccagcgaaacgttgctggataagctgattacccggctggcggctagcctgaaaagtccctttgcgctggataaattttgtgatgaggcatcgtgcagtgagcgcgttttgcgtcagcaatttcgccagcagactggaatgaccatcaatcaatatctgcgacaggtcagagtgtgtcatgcgcaatatcttctccagcatagccgcctgttaatcagtgatatttcgaccgaatgtggctttgaagatagtaactatttttcggtggtgtttacccgggaaaccgggatgacgcccagccagtggcgtcatctcaattcgcagaaagat

SEQ ID NO: 10 - DNA, 36 bp, synthetic construct
  misc_feature 1..36: note = RhaR extended
  source 1..36: mol_type = other DNA
agacgaaagggcctcgtgatacgcctatttttatag

SEQ ID NO: 11 - DNA, 837 bp, synthetic construct
  misc_feature 1..837: note = RhaS transcription activator
  source 1..837: mol_type = other DNA
atgaccgtattacatagtgtggatttttttccgtctggtaacgcgtccgtggcgatagaaccccggctcccgcaggcggattttcctgaacatcatcatgattttcatgaaattgtgattgtcgaacatggcacgggtattcatgtgtttaatgggcagccctataccatcaccggtggcacggtctgtttcgtacgcgatcatgatcggcatctgtatgaacataccgataatctgtgtctgaccaatgtgctgtatcgctcgccggatcgatttcagtttctcgccgggctgaatcagttgctgccacaagagctggatgggcagtatccgtctcactggcgcgttaaccacagcgtattgcagcaggtgcgacagctggttgcacagatggaacagcaggaaggggaaaatgatttaccctcgaccgccagtcgcgagatcttgtttatgcaattactgctcttgctgcgtaaaagcagtttgcaggagaacctggaaaacagcgcatcacgtctcaacttgcttctggcctggctggaggaccattttgccgatgaggtgaattgggatgccgtggcggatcaattttctctttcactgcgtacgctacatcggcagcttaagcagcaaacgggactgacgcctcagcgatacctgaaccgcctgcgactgatgaaagcccgacatctgctacgccacagcgaggccagcgttactgacatcgcctatcgctgtggattcagcgacagtaaccacttttcgacgctttttcgccgagagtttaactggtcaccgcgtgatattcgccagggacgggatggctttctgcaataa

SEQ ID NO: 12 - DNA, 816 bp, synthetic construct
  misc_feature 1..816: note = KanR
  source 1..816: mol_type = other DNA
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 13 - DNA, 105 bp, synthetic construct
  misc_feature 1..105: note = AmpR promoter
  source 1..105: mol_type = other DNA
cgcggaacccctatttgtttatttttctaaatacattcaaatatgtatccgctcatgagacaataaccctgataaatgcttcaataatattgaaaaaggaagagt

SEQ ID NO: 14 - DNA, 87 bp, synthetic construct
  misc_feature 1..87: note = rrnB T1 terminator
  source 1..87: mol_type = other DNA
caaataaaacgaaaggctcagtcgaaagactgggcctttcgttttatctgttgtttgtcggtgaacgctctcctgagtaggacaaat

SEQ ID NO: 15 - DNA, 28 bp, synthetic construct
  misc_feature 1..28: note = rrnB T2 terminator
  source 1..28: mol_type = other DNA
agaaggccatcctgacggatggcctttt

SEQ ID NO: 16 - DNA, 589 bp, synthetic construct
  misc_feature 1..589: note = pMB1 origin of replication
  source 1..589: mol_type = other DNA
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 17 - DNA, 6044 bp, synthetic construct
  misc_feature 1..6044: note = KTXHIS-Cert-Pelb1-LC-Pelb2-HC
  source 1..6044: mol_type = other DNA
ggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagactaggcttccgcgccctcatccgaaagggcgtattcatatatgcggtgttaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccaataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcatcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcttgtcagtaacgagaaggtcgcgaatccaggcgctttttagactggtcgtaatgaaattcaggaggaattgctcatgaagtatcttctgccgaccgcagcagcgggtctgctgctgctggcagcacagcctgcaatggcagatattcagatgactcagagcccaagttcgctgagcgcttctgttggcgatcgtgtgaccattacatgcaaagcctcacagaacgttggtaccaatgtcgcctggtatcagcagaaacctggaaaagcgcccaaagcgctcatctactcagcgagcttcctgtattcaggcgtgccgtatcgctttagcggctctggttccggtacagactttaccctcacgatttcgtccttacaaccggaagatttcgccacgtactattgccagcaatacaacatctatccgctgacctttggacaaggcaccaaagtggagatcaaacgcactgttgctgcaccgagtgtgttcatctttccaccgtctgatgagcagctgaagtctggtacagcaagtgttgtgtgtctgctgaacaacttctatccgcgtgaagctaaagtacagtggaaagtcgacaatgccttgcaatccgggaatagccaggaaagcgtgactgaacaggacagcaaggattcgacctacagtctgagcagtaccttaaccttgtcgaaagcggattacgagaaacacaaggtctatgcctgtgaagtcacgcatcaaggcctgtcatcgcctgttactaaatcatttaatagaggagaatgttaaatgaagtatctgttgccgactgctgcagcgggactgctgctgttagcggcacaaccggcgatggcggaagtgcagcttgtggagtctggaggtggcttagtccagccaggtggttccctgcgcttgtcctgtgcagcgagcgggtatgtattcacagattatggcatgaactgggttcggcaagcaccaggcaaaggcctcgaatggatggggtggatcaacacgtatattggggaaccgatttatgcggatagcgtcaaaggtcgcttcacgttcagtctggataccagcaaatcaaccgcgtatctccagatgaatagcctccgtgctgaagatactgccgtgtactactgtgcgcgtggttatcgcagttatgcgatggattactggggccaaggcaccttagtcaccgttagttctgcctccaccaaaggcccatcagtgtttccgctggccccttcgtctaaatcgacgagtggtggcacagccgcactgggatgcctggtcaaagactactttcccgaacctgtaaccgtaagctggaatagtggtgctttgacctcaggcgtgcatacgtttccggctgtcctgcagtcatccggtctgtactcgctttcgagcgttgttactgtaccctctagctccctgggcacccagacgtacatctgcaatgtgaaccataagccgtcgaacaccaaagtggacaagaaagttgagccgaaaagctgcgacaaaacgcacacatgtgccgcctaataaaagctt

SEQ ID NO: 18 - DNA, 66 bp, synthetic construct
  misc_feature 1..66: note = PelB1 signal peptide - LC - DNA
  source 1..66: mol_type = other DNA
atgaagtatcttctgccgaccgcagcagcgggtctgctgctgctggcagcacagcctgcaatggca

SEQ ID NO: 19 - DNA, 66 bp, synthetic construct
  misc_feature 1..66: note = PelB2 signal peptide - HC - DNA
  source 1..66: mol_type = other DNA
atgaagtatctgttgccgactgctgcagcgggactgctgctgttagcggcacaaccggcgatggcg

SEQ ID NO: 20 - DNA, 15 bp, synthetic construct
  misc_feature 1..15: note = TIR-LC
  source 1..15: mol_type = other DNA
ttgctcatgaagtat

SEQ ID NO: 21 - DNA, 15 bp, synthetic construct
  misc_feature 1..15: note = TIR-HC
  source 1..15: mol_type = other DNA
tgttaaatgaagtat

SEQ ID NO: 22 - DNA, 1615 bp, synthetic construct
  misc_feature 1..1615: note = RhaB frameshift - rhaBfs
  source 1..1615: mol_type = other DNA
tgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaaattcagcaggatcacattatgacctttcgcaattgtgtcgccgtcgatctcggcgcatccagtgggcgcgtgatgctggcgcgttacgagcgtgaatgccgcagcctgacgctgcgcgaaatccatcgttttaacaatgggctgcatagtcagaacggctatgtcacctgggatgtggatagcctggaaagtgccattcgccttggattaaacaaggtgtgcgaggaagggattcgtatcgcgatagcattgggattgatacctggggcgtggactttgtgctgctcgaccaacagggtcagcgtgtgggcctgcccgttgcttatcgcgatagccgcaccaatggcctaatggcgcaggcacaacaacaactcggcaaacgcgatatttatcaacgtagcggcatccagtttctgcccttcaatacgctttatcagttgcgtgcgctgacggagcaacaacctgaacttattccacacattgctcacgctctgctgatgccggattacttcagttatcgcctgaccggcaagatgaactgggaatataccaacgccacgaccacgcaactggtcaatatcaatagcgacgactgggacgagtcgctactggcgtggagcggggccaacaaagcctggtttggtcgcccgacgcatccgggtaatgtcataggtcactggatttgcccgcagggtaatgagattccagtggtcgccgttgccagccatgataccgccagcgcggttatcgcctcgccgttaaacggctcacgtgctgcttatctctcttctggcacctggtcattgatgggcttcgaaagccagacgccatttaccaatgacacggcactggcagccaacatcaccaatgaaggcggggcggaaggtcgctatcgggtgctgaaaaatattatgggcttatggctgcttcagcgagtgcttcaggagcagcaaatcaacgatcttccggcgcttatctccgcgacacaggcacttccggcttgccgcttcattatcaatcccaatgacgatcgct

SEQ ID NO: 23 - DNA, 1018 bp, synthetic construct
  misc_feature 1..1018: note = degP deletion
  source 1..1018: mol_type = other DNA
atataaaaatgtcgctgtaaaacatgtgtttagccatccagatgtcgagcggcttgaattgcagggctatcgggtcattagcggattattagagatttatcgtcctttattaagcctgtcgttatcagactttactgaactggtagaaaaagaacgggtgaaacgtttccctattgaatcgcgcttattccacaaactctcgacgcgccatcggctggcctatgtcgaggctgtcagtaaattaccgtcagattctcctgagtttccgctatgggaatattattaccgttgccgcctgctgcaggattatatcagcggtatgaccgacctctatgcgtgggatgaataccgacgtctgatggccgtagaacaataaccaggcttttgtaaagacgaacaataaatttttaccttttgcagaaactttagttcggaacttcaggctataaaacgaatctgaagaacacagcaattttgcgttatctgttaatcgagactgaaatacatgatctacctgttaatgcagtaatctccctcaaccccttcctgaaaacgggaaggggttctccttacaatctgtgaacttcaccacaactccatacatcttcatcatcctttaggcatttgcacaatgccgtacgttacgtacttccttatgctaagccgtgcataacggaggacttatggctggctggcatcttgataccaaaatggcgcaggatatcgtggcacgtaccatgcgcatcatcgataccaatatcaacgtaatggatgcccgtgggcgaattatcggcagcggcgatcgtgagcgtattggtgaattgcacgaaggtgcattgctggtactttcacagggacgagtcgtcgatatcgatgacgcggtagcacgtcatctgcacggtgtgcggcaggggattaatctaccgttacggctggaaggtgaaattgtcggcgtaattggcctgacaggtgaaccagagaatctgcgtaaatatggcgaactggtctgcatgacggc

SEQ ID NO: 24 - DNA, 1020 bp, synthetic construct
  misc_feature 1..1020: note = prc deletion
  source 1..1020: mol_type = other DNA
tttacggtgttaaacccggcgcaacgcgtgtcgatcttgacggcaacccatgcggtgagctggacgagcaacatgtagagcatgctcgcaagcagcttgaagaagcgaaagcgcgtgttcaggcacagcgtgctgaacagcaagcgaaaaaacgcgaagctgccgcaactgctggtgagaaagaagacgcaccgcgccgcgaacgcaagccacgtccgactacgccacgccgcaaagaaggcgctgaacgtaaacctcgtgcgcaaaagccggtagagaaagcgccaaaaacagtaaaagcacctcgcgaagaacagcacaccccggtttctgacatttcagctctgactgtcggacaagccctgaaggtgaaagcgggtcaaaacgcgatggatgccaccgtattagaaatcaccaaagacggcgtccgcgtccagctgaattcgggtatgtctttgattgtgcgcgcagaacacctggtgttctgaaacggaggccgggccaggcatgcaacccgctcccgtcaagtaatatcaatcaggcacaagaaattgtgcctgattttttaacagcgacaagatgccgtaaatcagatgctacaaaatgtaaagttgtgtctttctggtgacttacgcactatccagacttgaaaatagtcgcgtaacccatacgatgtgggtatcgcatattgcgttttgttaaactgaggtaaaaagaaaattatgatgcgaatcgcgctcttcctgctaacgaacctggccgtaatggtcgttttcgggctggtactgagcctgacagggatacagtcgagcagcgttcaggggctgatgatcatggccttgctgttcggttttggtggttccttcgtttcgcttctgatgtccaaatggatggcattacgatctgttggcggggaagtgatcgagcaaccgcgtaacgaaagggaacgttggctggtcaatactgtagcaacccaggctcgtcaggcggggatcgctatgccgcaagtggctatctacc

SEQ ID NO: 25 - DNA, 1064 bp, synthetic construct
  misc_feature 1..1064: note = sprW148R
  source 1..1064: mol_type = other DNA
aacaaacaacatggtcaaatctcaaccgattttgagatatatcttgcgcgggattcccgcgattgcagtagcggttctgctttctgcatgtagtgcaaataacaccgcaaagaatatgcatcctgagacacgtgcagtgggtagtgaaacatcatcactgcaagcttctcaggatgaatttgaaaacctggttcgtaatgtcgacgtaaaatcgcgaattatggatcagtatgctgactggaaaggcgtacgttatcgtctgggcggcagcactaaaaaaggtatcgattgttctggtttcgtacagcgtacattccgtgagcaatttggcttagaacttccgcgttcgacttacgaacagcaggaaatgggtaaatctgtttcccgcagtaatttgcgtacgggtgatttagttctgttccgtgccggttcaacgggacgccatgtcggtatttatatcggcaacaatcagtttgtccatgcttccaccagcagtggtgttattatttccagcatgaatgaaccgtacaggaagaagcgttacaacgaagcacgccgggttctcagccgcagctaataaaccgtttggatgcaatcccttggctatcctgacgagttaactgaaagcactgcttaggcagtgcttttttgttttcattcatcagagaaaatgatgtttccgcgtcttgatccaggctatagtccggtcattgttatcttttaaatgttgtcgtaatttcaggaaattaacggaatcatgttcatacgcgctcccaattttggacgtaagctcctgcttacctgcattgttgcaggcgtaatgattgcgatactggtgagttgccttcagtttttagtggcctggcataagcacgaagtcaaatacgacacactgattaccgacgtacaaaagtatctcgatacctattttgccgacctgaaatccactactgaccggctccagccgctgaccttagatacctgccagcaagctaaccccgaactgaccgcccgcgcagcgtttagcatgaatgtccgaacgtttgtgctggtgaaagataaaa

(19) United States
(12) Patent Application Publication
(10) Pub. No.: US 2025/0368732 A1
(43) Pub. Date: Dec. 4, 2025

(54) DNA CONSTRUCTS AND HOST CELLS FOR EXPRESSING RECOMBINANT PROTEIN

(71) Applicant: Alvotech HF, Reykjavik (IS)

(72) Inventors: Kiavash Mirzadeh, Solna (SE); David Vikström, Upplands Väsby (SE); Nurzian Ismail, Stockholm (SE); Patrik Samuelson, Saltsjöbaden (SE); Mariusz Baraszkiewicz, Bromma (SE); Tagrid Salih, Bromma Kyrka (SE); Kathleen Szekér, Marburg (DE); Daniel Chui, Södertälje (SE); Santhosh Gudise, Farsta (SE); Per Edebrink, Stockholm (SE); Maria Kadow, Sollentuna (SE); Karin Stefansson, Åkersberga (SE); Kristin Strandberg, Stockholm (SE)

(73) Assignees: Xbrane Biopharma AB, Solna (SE); Alvotech HF, Reykjavik (IS)

(21) Appl. No.: 18/694,642

(22) PCT Filed: Sep. 23, 2022
(86) PCT No.: PCT/EP2022/076591

§ 371 Date: Mar. 22, 2024

(30) Foreign Application Priority Data
Sep. 24, 2021 (SE) 2130258-3
Sep. 24, 2021 (SE) 2130259-1
Sep. 24, 2021 (SE) 2130261-7
Sep. 24, 2021 (SE) 2130263-3
Sep. 24, 2021 (SE) 2130264-1
Sep. 24, 2021 (SE) 2130265-8

(51) Int. Cl. C07K 16/24; C12N 15/70

(52) U.S. Cl. CPC C07K 16/241; C12N 15/70; C07K 2317/24; C07K 2317/55; C07K 2319/02

(57) ABSTRACT

A DNA construct for expressing a recombinant protein, wherein the DNA construct comprises: —at least one of the nucleotide sequences of SEQ ID No 20 and 21, wherein a nucleotide sequence of SEQ ID 20 and 21 is a TIR sequence; and —a nucleotide sequence which encodes a signal peptide; and wherein a nucleotide sequence of SEQ ID No 20 and 21 comprises at least the first 9 nucleotides of said signal peptide encoding sequence.

### INCORPORATION BY REFERENCE OF SEQUENCE LISTING

The Substitute Sequence Listing in XML file, named as 43726_SubstituteSequenceListing.xml of 53,313 bytes, created on Jan. 14, 2025, and submitted to the United States Patent and Trademark Office via Patent Center, is incorporated herein by reference.

### TECHNICAL FIELD

The present invention relates to DNA construct suitable for expressing recombinant proteins in a bacterial host cell. The present invention further relates to a vector and bacterial host cell comprising the DNA construct as well as a method of producing said recombinant protein by exposing said bacterial host cell to rhamnose and thereby inducing expression of said recombinant protein.

### BACKGROUND OF THE INVENTION

The metabolism of rhamnose involves L-rhamnose being taken up into cells via the permease RhaT and then isomerized into L-rhamnulose by L-rhamnose isomerase (RhaA), and L-rhamnulose is then phosphorylated further by rhamnulokinase (RhaB) and finally hydrolyzed by rhamnulose-1-phosphate aldolase (RhaD) to give dihydroxyacetonephosphate and L-lactaldehyde [1]. The genes rhaA, rhaB and rhaD form an operon referred to as rhaBAD and are transcribed with the aid of the rhaBAD promoter [1]. In comparison with other systems, the rhamnose metabolism pathway is distinguished by the fact that two transcription activators known as RhaS and RhaR are required for regulation as explained below [1].

The rhaBAD operon is a positively regulated catabolic operon which transcribes above mentioned rhaB, rhaA and rhaD genes divergently from the rhaSR operon with approximately 240 bp of DNA separating their respective transcription start sites [1]. The rhaSR operon encodes RhaS and RhaR wherein each monomer of the dimeric RhaS and RhaR proteins contains two helix-turn-helix motifs and contacts two major grooves of DNA. RhaR regulates transcription of rhaSR by binding promoter DNA spanning −32 to −82 bases relative to the rhaSR transcription start site [1]. Subsequent to rhaSR expression, RhaS bind DNA upstream of the rhaBAD operon at −32 to −81 bases relative to the transcription start site to increase rhaBAD expression [1]. Furthermore, the rhaSR-rhaBAD intergenic region contains CRP binding sites at positions −92.5 (CRP 1) relative to the transcription start site of the rhaBAD operon and CRP binding sites at positions −92.5 (CRP 2), −115.5 (CRP 3) and −116.5 (CRP 4) relative to the transcription start site of the rhaSR operon [1]. The cyclic AMP receptor protein (CRP) regulates the expression of more than 100 promoters in Escherichia coli.

DNA constructs comprising DNA sequences encoding RhaS, RhaR and the rhaBAD promoter are known in the art. U.S. Pat. No. 8,138,324 discloses pTACO- and pLEMO-derived plasmids (i.e. DNA constructs) comprising DNA sequences encoding RhaS, RhaR and the rhaBAD promoter. However, U.S. Pat. No. 8,138,324 is silent about using host cells which have a disabled rhamnose metabolism.

DNA constructs based on pRha-derived plasmids comprising DNA sequences encoding RhaS, RhaR and the rhaBAD promoter are also known in the art, for example from Giacalone et al. [5] or Hjelm et al. [2]. Giacalone et al. describe for example the plasmids pRha67A and pRha109A whereas Hjelm et al. disclose the plasmid pRha67K.

Although DNA constructs comprising DNA sequences encoding RhaS, RhaR and the rhaBAD promoter are known in the art there are still many challenges, especially in industrial scale production of recombinant proteins, in particular monoclonal antibodies or fragments thereof. The main challenges are:
(i) the host cells being subject to stress which results in damage to cellular macromolecules such as membranes, proteins and nucleic acids;
(ii) poor growth of the host cells;
(iii) poor activity of the produced recombinant proteins; and/or
(iv) obtaining recombinant proteins in low yields.

Hence, there is a need for improved DNA constructs as well as a host cell and method suitable for the efficient production of recombinant proteins, such as monoclonal antibodies or fragments thereof in a high yield.

In particular, there is a need for improved DNA constructs as well as a host cell for the efficient production of Certolizumab which is a humanized Fab′ fragment (from an IgG 1 isotype) of an anti-tumor necrosis factor (TNF) monoclonal antibody with affinity for TNF-alpha. The conjugation of Certolizumab with an approximately 40 kDa polyethylene glycol (PEG) results in Certolizumab pegol which is a pharmaceutical marketed by UCB as Cimzia® and which is administered by subcutaneous injection for the treatment of Crohn's disease, rheumatoid arthritis, psoriatic arthritis and ankylosing spondylitis.

Patents such as EP1287140 and U.S. Pat. No. 7,012,135 disclose DNA constructs for the production of Certolizumab. However, these DNA constructs, which comprise un-evolved translation initiation regions (TIRs) and which furthermore appear to lack nucleotide sequences encoding PelB signal peptide, are not optimal for producing Certolizumab with high yields.

Typically, when generating recombinant expression vectors such as in the ones disclosed in EP1287140 and U.S. Pat. No. 7,012,135, the TIR is formed by fusing the 5′UTR (i.e. untranslated region upstream of the ATG start codon) from the expression vector with the coding sequence of a signal peptide. Each time a different signal peptide is used, a different TIR is generated. Such TIRs are referred to as un-evolved as they were formed by ad hoc genetic fusion rather than the synthetically evolved TIRs described in the present invention as well as in U.S. Pat. No. 10,696,963 and WO21158163.

Patents such as U.S. Pat. No. 6,828,121 and EP1341899 relate to host cells for the production of various types of antibodies and antibody fragments such as humanized Fab′ fragments. Some specific examples of antibodies which can be produced by these host cells are anti-IgE, anti-IgG, anti-Her-2, anti-CD11a, anti-CD18, anti-CD20 and anti-VEGF. An example of a host cell disclosed in U.S. Pat. No. 6,828,121 and EP1341899 is an E. coli strain deficient in chromosomal degP and prc encoding protease DegP and Prc, respectively, and harboring a mutant spr gene, wherein the product of the mutant spr gene is characterized by the tryptophan at position 148 being changed to arginine. However, U.S. Pat. No. 6,828,121 and EP1341899 are both silent about (a) mutations of the host cell relating to the metabolism of rhamnose, and (b) production of a specific Fab′ fragment such as Certolizumab.

International patent application WO21158163 relates to DNA constructs comprising synthetically evolved TIRs for regulating the performance of signal peptides in the production of recombinant proteins. WO21158163 clearly shows that synthetically evolved TIRs have technical advantages over un-evolved TIRs. However, WO21158163 is silent about synthetically evolved TIRs specifically developed for the optimal expression of Certolizumab.

WO21158163 further relates to nucleotide sequences for the expression of the Pelb signal peptide. However, WO21158163 is silent about nucleotide sequences specifically developed for the optimal expression of Certolizumab.

Hence, there is a need for optimization of DNA constructs, host cells, TIRs and signal peptides for the expression of recombinant proteins such as Certolizumab.

### OBJECTS OF THE INVENTION

The object of the present invention is to provide advantageous technical effect of DNA constructs.

A further object of the present invention is to provide advantageous technical effect of TIRs.

A further object of the present invention is to provide advantageous technical effect of the host cells.

A further object of the present invention is to provide advantageous technical effect of signal peptide nucleotide sequences.

A further object of the present invention is to provide advantageous methods for the efficient production of recombinant proteins.

### SUMMARY OF THE INVENTION

The objects of the invention have been attained by any one or more of the below disclosed aspects of the invention.

A first aspect of the invention relates to a DNA construct suitable for expressing Certolizumab in a host cell, wherein Certolizumab comprises (i) a light chain comprising the amino acid sequence of SEQ ID 3, and (ii) a heavy chain comprising the amino acid sequence of SEQ ID 4,
wherein said DNA construct comprises a nucleotide sequence encoding Certolizumab,
wherein said DNA construct further comprises at least one nucleotide sequence encoding a signal peptide which is operably linked in the direction of transcription to the nucleotide sequence encoding the light chain of Certolizumab and/or the heavy chain of Certolizumab,
wherein said DNA construct further comprises nucleotide sequences encoding:
   a promoter,
   RhaR transcription activator,
   RhaS transcription activator,
   an antibiotic resistance marker,
   at least one terminator, and
   an origin of replication.

In a preferred embodiment, the DNA construct is characterized by:
a promoter,
RhaR transcription activator,
RhaS transcription activator,
an antibiotic resistance marker,
a promoter operably linked to the nucleotide sequence encoding the antibiotic resistance marker,
at least one terminator, and
an origin of replication.

In a preferred embodiment, the DNA construct is characterized by:
rhaBAD promoter,
RhaR transcription activator,
RhaS transcription activator,
an antibiotic resistance marker,
a promoter operably linked to the nucleotide sequence encoding the antibiotic resistance marker,
rrnB T1 terminator,
rrnB T2 terminator, and
an pMB1 origin of replication.

In a preferred embodiment, the DNA construct is characterized by:
the antibiotic resistance marker is a kanamycin resistance marker, preferably a kanamycin resistance marker comprising the nucleotide sequence of SEQ ID 12 or a sequence with at least 90% sequence identity thereto;
the promoter operably linked to the nucleotide sequence encoding the antibiotic resistance marker is an AmpR promoter, preferably an AmpR promoter comprising the nucleotide sequence of SEQ ID 13 or a sequence with at least 90% sequence identity thereto;
the rrnB T1 terminator comprising the nucleotide sequence of SEQ ID 14 or a sequence with at least 90% sequence identity thereto;
the rrnB T2 terminator comprising the nucleotide sequence of SEQ ID 15 or a sequence with at least 90% sequence identity thereto; and/or
the pMB1 origin of replication comprising the nucleotide sequence of SEQ ID 16 or a sequence with at least 90% sequence identity thereto.

In a preferred embodiment, the DNA construct is characterized by:
the antibiotic resistance marker being a kanamycin resistance marker comprising the nucleotide sequence of SEQ ID 12;
the promoter operably linked to the nucleotide sequence encoding the antibiotic resistance marker is an AmpR promoter comprising the nucleotide sequence of SEQ ID 13;
the rrnB T1 terminator comprising the nucleotide sequence of SEQ ID 14;
the rrnB T2 terminator comprising the nucleotide sequence of SEQ ID 15; and/or
the pMB1 origin of replication comprising the nucleotide sequence of SEQ ID 16.

In a preferred embodiment, the DNA construct is characterized by:
the rhaBAD promoter comprising the nucleotide sequence of SEQ ID 8 or a sequence with at least 90% sequence identity thereto;
the RhaR transcription activator comprising the nucleotide sequence of SEQ ID 9 or a sequence with at least 90% sequence identity thereto;
the RhaS transcription activator comprising the nucleotide sequence of SEQ ID 11 or a sequence with at least 90% sequence identity thereto;
the antibiotic resistance marker is a kanamycin resistance marker comprising the nucleotide sequence of SEQ ID 12 or a sequence with at least 90% sequence identity thereto;
the AmpR promoter comprising the nucleotide sequence of SEQ ID 13 or a sequence with at least 90% sequence identity thereto;
the rrnB T1 terminator comprising the nucleotide sequence of SEQ ID 14 or a sequence with at least 90% sequence identity thereto;
the rrnB T2 terminator comprising the nucleotide sequence of SEQ ID 15 or a sequence with at least 90% sequence identity thereto; and
the pMB1 origin of replication comprising the nucleotide sequence of SEQ ID 16 or a sequence with at least 90% sequence identity thereto.

In a preferred embodiment, the DNA construct is characterized by:
the rhaBAD promoter comprising the nucleotide sequence of SEQ ID 8;
the RhaR transcription activator comprising the nucleotide sequence of SEQ ID 9;
the RhaS transcription activator comprising the nucleotide sequence of SEQ ID 11;
the antibiotic resistance marker is a kanamycin resistance marker comprising the nucleotide sequence of SEQ ID 12;
the AmpR promoter comprising the nucleotide sequence of SEQ ID 13;
the rrnB T1 terminator comprising the nucleotide sequence of SEQ ID 14;
the rrnB T2 terminator comprising the nucleotide sequence of SEQ ID 15; and
the pMB1 origin of replication comprising the nucleotide sequence of SEQ ID 16.

In a preferred embodiment, the DNA construct may comprise one or more restriction sites cleavable by restriction enzymes such as EcoRI, NdeI, NotI, XhoI, PspXI, PaeR71, BbsI, StyI, AvrII, BanI, Acc65I, KpnI, Eco53kI, SacI, BamHI, XbaI, SalI, AccI, PstI, SbfI, SphI and/or HindIII.

In a preferred embodiment, the DNA construct further comprises a nucleotide sequence encoding said recombinant protein operably linked to the rhaBAD promoter, wherein said recombinant protein is a monoclonal antibody or fragment thereof, preferably said recombinant protein is Certolizumab. More preferably said recombinant protein is Certolizumab comprising (i) a light chain comprising the amino acid sequence of SEQ ID 3, and/or (ii) a heavy chain comprising the amino acid sequence of SEQ ID 4.

In a preferred embodiment, the DNA construct comprises a nucleotide sequence encoding the recombinant protein operably linked to the rhaBAD promoter comprising (i) a nucleotide sequence encoding for the light chain of Certolizumab comprising the sequence of SEQ ID 5 or a sequence with at least 90% sequence identity thereto, and/or (ii) a nucleotide sequence encoding for the heavy chain of Certolizumab comprising the sequence of SEQ ID 6 or a sequence with at least 90% sequence identity thereto; preferably said nucleotide sequence encoding the recombinant protein comprises (i) a nucleotide sequence encoding for the light chain of Certolizumab comprising the sequence of SEQ ID 5, and/or (ii) a nucleotide sequence encoding for the heavy chain of Certolizumab comprising the sequence of SEQ ID 6.

In an embodiment, the DNA construct further comprises a nucleotide sequence encoding the recombinant protein operably linked to the rhaBAD promoter comprising at least one nucleotide sequence encoding a signal peptide which is operably linked in the direction of transcription to either one or both of the nucleotide sequence of SEQ ID 5 and SEQ ID 6, preferably the signal peptide is a PelB (pectate lyase B) signal peptide.

The nucleotide sequence encoding the PelB signal peptide which is operably linked in the direction of transcription to the nucleotide sequence of the light chain of Certolizumab is in the present invention referred to as PelB1. The nucleotide sequence of PelB1 comprises a sequence of SEQ ID 18 or a sequence with at least 90% sequence identity thereto.

The nucleotide sequence encoding the PelB signal peptide which is operably linked in the direction of transcription to the nucleotide sequence of the heavy chain of Certolizumab is in the present invention referred to as PelB2. The nucleotide sequence of PelB2 comprises a sequence of SEQ ID 19 or a sequence with at least 90% sequence identity thereto.

The resulting PelB signal peptide comprises an amino acid sequence of SEQ ID 7 [6]: MKYLLPTAAAGLLLLAAQPAMA.

In an embodiment, the DNA construct comprises a TIR having a nucleotide sequence of SEQ ID 20, wherein said sequence of SEQ ID 20 comprises at least the first 9 nucleotides of the nucleotide sequence of PelB1, i.e. the first 9 nucleotides of SEQ ID 18. This particular TIR is in the present invention also referred to as TIR-LC.

In an embodiment, the DNA construct comprises a TIR having a sequence of SEQ ID 21, wherein said sequence of SEQ ID 21 comprises at least the first 9 nucleotides of the nucleotide sequence of PelB2, i.e. the first 9 nucleotides of SEQ ID 19. This particular TIR is in the present invention also referred to as TIR-HC.

In a preferred embodiment, the DNA construct comprises the sequence of SEQ ID 17 or a sequence with at least 90% sequence identity thereto, preferably comprises the sequence of SEQ ID 17.

A second aspect of the invention relates to a DNA construct for expressing a recombinant protein, wherein the DNA construct comprises:
at least one of the nucleotide sequence of SEQ ID No 20 and 21, wherein a nucleotide sequence of SEQ ID 20 and 21 is a TIR sequence; and
a nucleotide sequence which encodes a signal peptide;
   and wherein a nucleotide sequence of SEQ ID No 20 and 21 comprises at least the first 9 nucleotides of said signal peptide encoding sequence.

In an embodiment, the DNA construct comprises a TIR having a nucleotide sequence of SEQ ID 20, wherein said sequence of SEQ ID 20 comprises at least the first 9 nucleotides of the nucleotide sequence of PelB1, i.e. the first 9 nucleotides of SEQ ID 18. This particular TIR is in the present invention also referred to as TIR-LC.

In an embodiment, the DNA construct comprises a TIR having a sequence of SEQ ID 21, wherein said sequence of SEQ ID 21 comprises at least the first 9 nucleotides of the nucleotide sequence of PelB2, i.e. the first 9 nucleotides of SEQ ID 19. This particular TIR is in the present invention also referred to as TIR-HC.

In an embodiment, the nucleotide sequence which encodes a signal peptide is operably linked to:
the first nucleotide sequence which encodes the light chain of an antibody; and/or
the second nucleotide sequence which encodes the heavy chain of an antibody.

In an embodiment, the DNA construct comprises a Shine-Dalgarno sequence. The Shine-Dalgarno sequence is located upstream from the ATG start codon of the nucleotide sequence which encodes a signal peptide. In an embodiment, said Shine-Dalgarno sequence is located upstream from the ATG start codon of the nucleotide sequence which encodes a signal peptide which is operably linked to the light and/or heavy chain of an antibody. In an embodiment, said Shine-Dalgarno sequence comprises nucleotide sequence AGGAGGAA and/or GAGGAGAA in the direction of transcription. Preferably, AGGAGGAA is upstream of nucleotide sequence coding for the light chain of an antibody. Preferably, GAGGAGAA is upstream of nucleotide sequence coding for the heavy chain of an antibody. More preferably, AGGAGGAA is upstream of TIR-LC. More preferably, GAGGAGAA is upstream of TIR-HC.

In an embodiment, the first nucleotide sequence which encodes a signal peptide (e.g. PelB1) is operably linked to the first nucleotide sequence which encodes the light chain of an antibody.

In an embodiment, the second nucleotide sequence which encodes a signal peptide (e.g. PelB2) is operably linked to the second nucleotide sequence which encodes the heavy chain of an antibody.

In an embodiment, the first and second nucleotide sequences which encode for the light and heavy chains of an antibody, respectively, encode amino acid sequences of SEQ ID No 3 and SEQ ID No 4, respectively.

In an embodiment, the first and second nucleotide sequence which encode the light and heavy chains of an antibody, respectively, comprise nucleotide sequences of SEQ ID No 5 and SEQ ID No 6, respectively.

A third aspect of the invention relates to a DNA construct for expressing a signal peptide, wherein the DNA construct comprises a nucleotide sequence which encodes a PelB signal peptide, wherein the nucleotide sequence which encodes said PelB signal peptide comprises at least one of the nucleotide sequences of SEQ ID No 18 and 19. In an embodiment, the DNA construct comprises both of the nucleotide sequences SEQ ID No 18 and 19.

A fourth aspect of the invention relates to DNA construct comprising nucleotide sequence encoding amino acid sequences, wherein the amino acid sequences comprise:
a. the amino acid sequence for Certolizumab;
b. a first signal peptide of amino acid sequence of SEQ ID No 7 fused to the N-terminus of light chain amino acid sequence of Certolizumab; and
c. a second signal peptide of amino acid sequence of SEQ ID NO 7 fused to the N-terminus of heavy chain amino acid sequence of Certolizumab.

In an embodiment, the nucleotide sequences which encodes said first and second signal peptides comprise nucleotide sequences of SEQ ID No 18 and 19, respectively.

A fifth aspect of the invention relates to an expression vector comprising any of the DNA constructs according to the first, second, third and/or fourth aspects of the invention.

A sixth aspect of the invention relates to a host cell characterized by a chromosome comprising:
a. a mutation in the nucleotide sequence encoding RhaB which disables rhamnose metabolism;
b. a mutation in the degP gene which disables (i) expression of DegP protease and/or (ii) activity of DegP protease;
c. a mutation in prc gene which disables (i) expression of Prc protease, and/or (ii) activity of Prc protease; and
d. a mutation in the spr gene.

In an embodiment, a mutation is selected from the group consisting of frameshift, deletion, substitution and insertion.

In an embodiment, said mutation in the nucleotide sequence encoding RhaB which disables rhamnose metabolism is a frame shift-mutation in the nucleotide sequence encoding RhaB.

In an embodiment, said mutation in the degP gene is a degP deletion.

In an embodiment, said mutation in the prc gene is a prc deletion.

In an embodiment, said mutation in the spr gene is a sprW148R mutation characterized by substitution in the spr gene resulting in tryptophan at position 148 being changed to arginine.

In an embodiment, said host cell characterized by a chromosome comprising:
a. a mutation in the nucleotide sequence encoding RhaB which disables rhamnose metabolism;
b. a mutation in the degP gene which disables expression of DegP protease;
c. a mutation in prc gene which disables expression of Prc protease; and
d. a mutation in the spr gene.

In an embodiment, said host cell is a bacterial cell, more preferably E. coli, most preferably E. coli W3110.

In an embodiment, said host cell is an E. coli W3110, comprising a chromosome which comprises a frame shift-mutation in the nucleotide sequence encoding RhaB. This particular host cell is in the present invention referred to as E. coli W3110 rhaBfs as well as XB17.

In an embodiment, said host cell is E. coli W3110 rhaBfs further comprising a chromosome which comprises a degP deletion. This particular host cell is in the present invention referred to as E. coli W3110 rhaBfs ΔDegP as well as XB83.

In an embodiment, said host cell is E. coli W3110 rhaBfs ΔDegP further comprising a chromosome which comprises a prc deletion. This particular host cell is in the present invention referred to as E. coli W3110 rhaBfs ΔdegP Δprc as well as XB152.

In an embodiment, said host cell is E. coli W3110 rhaBfs ΔdegP Δprc further comprising a chromosome which comprises a sprW148R mutation. This particular host cell is in the present invention referred to as E. coli W3110 rhaBfs ΔDegP Δprc sprW148R as well as XB166.

A seventh aspect of the invention relates to a host cell according to the sixth aspect of the invention comprising a DNA construct according to the first, second, third and/or fourth aspects of the invention.

An eighth aspect of the invention relates to a method of producing a recombinant protein comprising the step of exposing the host cell according to the seventh aspect of the invention to rhamnose, thereby inducing expression of said recombinant protein. In a preferred embodiment, the method further comprises the step of recovering the recombinant protein from the bacterial host cell; and optionally further comprises one or more step(s) of purifying the recovered recombinant protein, preferably by one or more chromatography steps.

A ninth aspect of the invention relates to a method of producing a recombinant protein, comprising the step of introducing the DNA construct according to the first, second, third and/or fourth aspects of the invention into a host cell according to the sixth aspect of the invention.

In an embodiment, the method further comprises the step of exposing the host cell to rhamnose, thereby inducing expression of the recombinant protein.

In an embodiment, the method further comprises the step of recovering the recombinant protein from the host cell; and optionally further comprises one or more step(s) of purifying the recovered recombinant protein, preferably by one or more chromatography steps.

In an embodiment, the method further comprises the step of derivatizing the purified recombinant protein, preferably with a polyethylene glycol moiety, more preferably with an about 40 kDa polyethylene glycol moiety.

A tenth aspect of the invention relates to a recombinant protein obtainable by a method according to the ninth aspect of the invention. The recombinant protein is preferably an antibody or a fragment thereof, more preferably a Fab′ fragment antibody, most preferably Certolizumab.

An eleventh aspect of the invention relates to a Certolizumab biosimilar obtainable by a method according to the ninth aspect of the invention. Preferably, the Certolizumab biosimilar comprises a polyethylene glycol moiety such as an about 40 kDa polyethylene glycol moiety. The Certolizumab biosimilar is here disclosed as a product-by-process in order to satisfactorily protect the molecular structure of the Certolizumab biosimilar. A biosimilar is a highly similar to the reference product, i.e. the Certolizumab biosimilar will have highly similar molecular structure and bioactivity as Certolizumab pegol (Cimzia®) which is produced by the reference product sponsor. Moreover, a biosimilar has no clinically meaningful differences from a reference product and the clinical trials that are conducted on biosimilars assess pharmacokinetics and immunogenicity. Nevertheless, the minor structural differences between a Certolizumab biosimilar according to the present invention and Certolizumab pegol of the reference product sponsor will partially be due to the method according to the ninth aspect of the invention (as well as the eight aspect of the invention).

A twelfth aspect of the invention relates to a Certolizumab biosimilar, or a derivative thereof, for use as medicament, preferably for use in the treatment of Crohn's disease, rheumatoid arthritis, psoriatic arthritis and ankylosing spondylitis. Said derivative preferably comprises a polyethylene glycol moiety such as an about 40 kDa polyethylene glycol moiety. An embodiment of the invention relates to a method of treating a disease by using a Certolizumab biosimilar. The disease may be Crohn's disease, rheumatoid arthritis, psoriatic arthritis and ankylosing spondylitis

A thirteenth aspect of the invention relates to a method of producing a signal peptide, comprising the step of introducing the DNA construct according to the first, second, third and/or fourth aspects of the invention into a host cell according to the sixth aspect of the invention.

One or more of the above indicated SEQ IDs 1-21 of the various aspects of the invention (and embodiments thereof) may in some embodiments be replaced by a sequence with at least 90% sequence identity thereto. The term “sequence identity” as used herein is used with regard to amino acid or nucleotide sequences and the sequence identity is over the entire length of the specified sequence. A sequence may thus be at least 90 percent, at least 92 percent, at least 95 percent, at least 96 percent, at least 97 percent, at least 98 percent or at least 99 percent, identical in sequence to the amino acid or nucleotide sequence specified. Such sequences of the invention thus include single or multiple nucleotide or amino acid alterations (additions, substitutions, insertions or deletions) to the sequences of the invention. At the amino acid level preferred sequences with the above defined sequence identity contain up to 5, e.g. only 1, 2, 3, 4 or 5, preferably 1, 2 or 3, more preferably 1 or 2, altered amino acids in the sequences of the invention.

### BRIEF DESCRIPTION OF DRAWINGS

FIG. 1—Plasmid map for KTXHIS

FIG. 2—The nucleotide sequence of the multiple cloning site (MCS) of KTXHIS

FIG. 3—Plasmid map of KTXHIS-Cert-PelB1-LC-PelB2-HC

FIG. 4—Example of TIR library selection and isolation of an evolved TIR therefrom

FIG. 5—Western-blot analysis of media fraction

FIG. 6—Western-blot analysis of total fraction

FIG. 7—NanoDrop™ and ÄKTA chromatography performed to detect yield and titer differences between expression systems XB62 (XB17 host cell containing expression vector D37 having un-evolved TIRs) and XB102 (XB17 host cell containing expression vector E83 having synthetically evolved TIR for the regulation of the Certolizumab heavy chain expression).

FIG. 8—A comparative expression analysis through periplasmic extraction followed by Affinity-HPLC: E83 expression vector expressed in XB17 host cell versus E83 vector expressed in XB166 host cell

FIG. 9—A comparative expression analysis through periplasmic extraction followed by Affinity-HPLC: E83 expression vector expressed in XB166 host cell versus E111 vector expressed in XB166 host cell

### DETAILED DESCRIPTION

A specific embodiment of the present invention relates to DNA constructs for the expression of antibody wherein said DNA construct comprises an improved TIR of SEQ ID 20:

TTGCTCATGAAGTAT

Another specific embodiment relates to DNA constructs for the expression of antibody wherein said DNA construct comprises an improved TIR of SEQ ID 21:

TGTTAAATGAAGTAT

The TIRs of SEQ ID 20 and 21 may be comprised in the same DNA construct. An example of such an embodiment is that the TIR of SEQ ID 20 is upstream of the nucleotide sequence expressing a light chain of an antibody or a fragment thereof (such as Certolizumab) while the TIR of SEQ ID 21 is upstream of the nucleotide sequence expressing a heavy chain of an antibody or a fragment thereof (such as Certolizumab).

A specific embodiment of the invention relates to an improved nucleotide sequence of SEQ ID 18 encoding a PelB signal peptide which is operably linked to the nucleotide sequences encoding a chain of an antibody:

ATGAAGTATCTTCTGCCGACCGCAGCAGCGGGTCTGCTGCTGCTGGCAGC
ACAGCCTGCAATGGCA

Another specific embodiment relates to an improved nucleotide sequence of SEQ ID 19 encoding a PelB signal peptide which is operably linked to the nucleotide sequences encoding a chain of an antibody:

ATGAAGTATCTGTTGCCGACTGCTGCAGCGGGACTGCTGCTGTTAGCGGC
ACAACCGGCGATGGCG

The PelB nucleotide sequence of SEQ ID 18 and 19 may be comprised in the same DNA construct. An example of such an DNA construct is when the PelB nucleotide sequence of SEQ ID 18 is operably linked to the nucleotide sequences encoding the light chain of an antibody or fragment thereof (such as Certolizumab) while the PelB nucleotide sequence of SEQ ID 19 is operably linked to the nucleotide sequences encoding the heavy chain of an antibody or fragment thereof (such as Certolizumab).

Yet in other specific embodiments, the above described sequences of SEQ ID 18-21 may be comprised in the same DNA construct. In such embodiments, a TIR nucleotide sequence of SEQ ID No 20 and 21 will comprises at least the first 9 nucleotides of a signal peptide nucleotide sequence of SEQ ID 18 and 19.

Other specific embodiments of present invention may relate to regulating the L-rhamnose rhaBAD promoter-based production of recombinant proteins such as Certolizumab. In other words, Certolizumab may be produced by:
a. cloning a nucleotide sequence encoding Certolizumab into a DNA construct such that the nucleotide sequence is operably linked to a rhaBAD promoter, and
b. introducing the resulting nucleotide sequence into a bacterial host cell comprising a chromosome which comprises a mutation or modification which disables rhamnose metabolism.

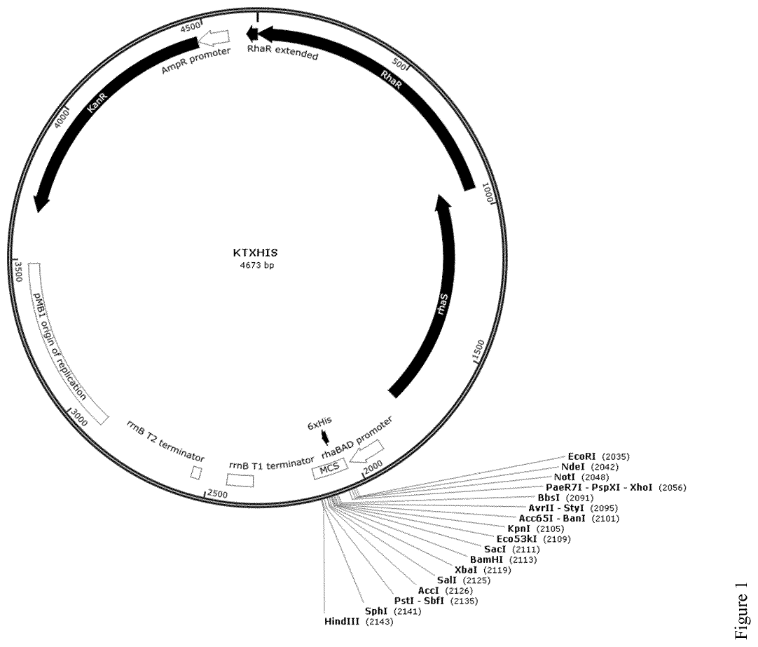

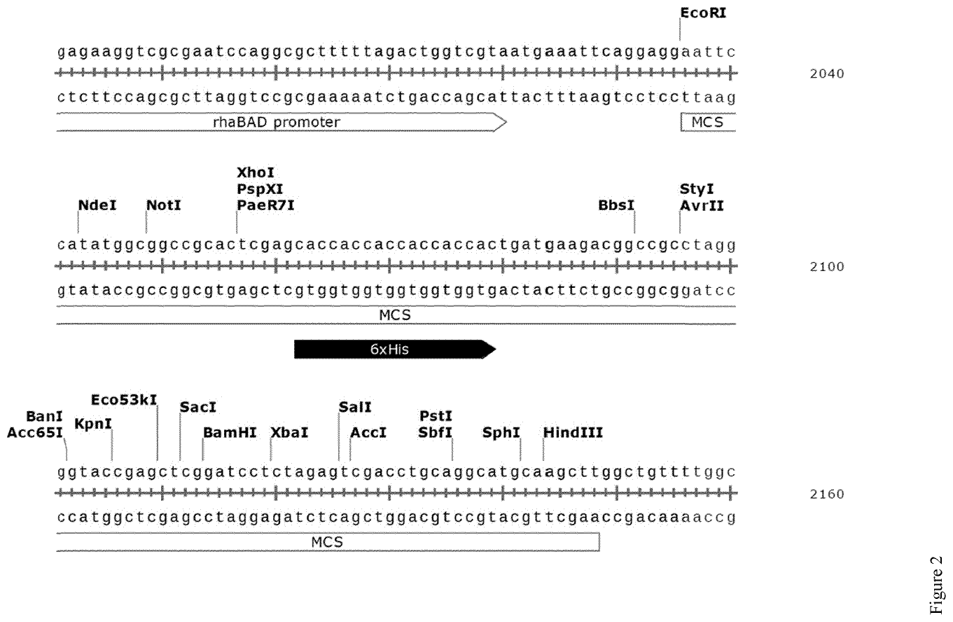

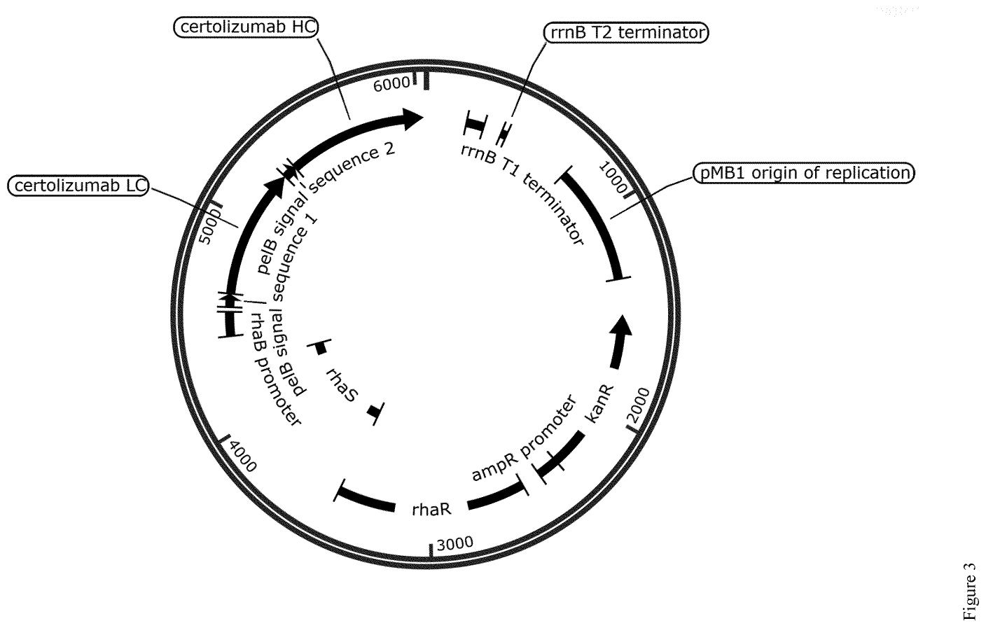

In an embodiment, the nucleotide sequence of the rhaBAD promoter comprises the sequence of SEQ ID 8 (and wherein the sequence is referred to as “rhaBAD” in FIGS. 1 and 3): CACCACAATTCAGCAAATTGTGAACATCATCACGTTCATCTTTCCCTGGTTGCC AATGGCCCATTTTCTTGTCAGTAACGAGAAGGTCGCGAATCCAGGCGCTTTTTAG ACTGGTCGTA.

The DNA construct may comprise a nucleotide sequence encoding the RhaR transcription activator. In an embodiment of the invention, the nucleotide sequence of the RhaR transcription activator comprises a sequence of SEQ ID 9 (and wherein the sequence is referred to as “rhaR” in FIGS. 1 and 3):

ATGGCTTTCTGCAATAACGCGAATCTTCTCAACGTATTTGTACGCCATAT
TGCGAATAATCAACTTCGTTCTCTGGCCGAGGTAGCCACGGTGGCGCATC
AGTTAAAACTTCTCAAAGATGATTTTTTTGCCAGCGACCAGCAGGCAGTC
GCTGTGGCTGACCGTTATCCGCAAGATGTCTTTGCTGAACATACACATGA
TTTTTGTGAGCTGGTGATTGTCTGGCGCGGTAATGGCCTGCATGTACTCA
ACGATCGCCCTTATCGCATTACCCGTGGCGATCTCTTTTACATTCATGCT
GATGATAAACACTCCTACGCTTCCGTTAACGATCTGGTTTTGCAGAATAT
TATTTATTGCCCGGAGCGTCTGAAGCTGAATCTTGACTGGCAGGGGGCGA
TTCCGGGATTTAACGCCAGCGCAGGGCAACCACACTGGCGCTTAGGTAGC
ATGGGGATGGCGCAGGCGCGGCAGGTTATTGGTCAGCTTGAGCATGAAAG
TAGTCAGCATGTGCCGTTTGCTAACGAAATGGCTGAGTTGCTGTTCGGGC
AGTTGGTGATGTTGCTGAATCGCCATCGTTACACCAGTGATTCGTTGCCG
CCAACATCCAGCGAAACGTTGCTGGATAAGCTGATTACCCGGCTGGCGGC
TAGCCTGAAAAGTCCCTTTGCGCTGGATAAATTTTGTGATGAGGCATCGT
GCAGTGAGCGCGTTTTGCGTCAGCAATTTCGCCAGCAGACTGGAATGACC
ATCAATCAATATCTGCGACAGGTCAGAGTGTGTCATGCGCAATATCTTCT
CCAGCATAGCCGCCTGTTAATCAGTGATATTTCGACCGAATGTGGCTTTG
AAGATAGTAACTATTTTTCGGTGGTGTTTACCCGGGAAACCGGGATGACG
CCCAGCCAGTGGCGTCATCTCAATTCGCAGAAAGAT.

The DNA construct may further comprise a nucleotide sequence encoding an extension of the RhaR transcription activator which is in frame with RhaR because of a missing stop codon. In an embodiment of the invention, the nucleotide sequence of the extension of the RhaR transcription activator comprises the sequence of SEQ ID 10 (and wherein the sequence is referred to as “rhaR extended” in FIGS. 1 and 3):

AGACGAAAGGGCCTCGTGATACGCCTATTTTTATAG.

The DNA construct may comprise a nucleotide sequence encoding the RhaS transcription activator. In an embodiment of the invention, the nucleotide sequence of the RhaS transcription activator comprises the sequence of SEQ ID 11 (and wherein the sequence is referred to as “rhaS” in FIGS. 1 and 3):

ATGACCGTATTACATAGTGTGGATTTTTTTCCGTCTGGTAACGCGTCCGT
GGCGATAGAACCCCGGCTCCCGCAGGCGGATTTTCCTGAACATCATCATG
ATTTTCATGAAATTGTGATTGTCGAACATGGCACGGGTATTCATGTGTTT
AATGGGCAGCCCTATACCATCACCGGTGGCACGGTCTGTTTCGTACGCGA
TCATGATCGGCATCTGTATGAACATACCGATAATCTGTGTCTGACCAATG
TGCTGTATCGCTCGCCGGATCGATTTCAGTTTCTCGCCGGGCTGAATCAG
TTGCTGCCACAAGAGCTGGATGGGCAGTATCCGTCTCACTGGCGCGTTAA
CCACAGCGTATTGCAGCAGGTGCGACAGCTGGTTGCACAGATGGAACAGC
AGGAAGGGGAAAATGATTTACCCTCGACCGCCAGTCGCGAGATCTTGTTT
ATGCAATTACTGCTCTTGCTGCGTAAAAGCAGTTTGCAGGAGAACCTGGA
AAACAGCGCATCACGTCTCAACTTGCTTCTGGCCTGGCTGGAGGACCATT
TTGCCGATGAGGTGAATTGGGATGCCGTGGCGGATCAATTTTCTCTTTCA
CTGCGTACGCTACATCGGCAGCTTAAGCAGCAAACGGGACTGACGCCTCA
GCGATACCTGAACCGCCTGCGACTGATGAAAGCCCGACATCTGCTACGCC
ACAGCGAGGCCAGCGTTACTGACATCGCCTATCGCTGTGGATTCAGCGAC
AGTAACCACTTTTCGACGCTTTTTCGCCGAGAGTTTAACTGGTCACCGCG
TGATATTCGCCAGGGACGGGATGGCTTTCTGCAATAA.

The DNA construct may comprise a nucleotide sequence encoding an “antibiotic resistance marker” or “selection marker”. Such a marker is a fragment of DNA that contains a gene whose product confers resistance to an antibiotic (e.g., chloramphenicol, ampicillin, gentamycin, streptomycin, tetracycline, kanamycin, neomycin) or the ability to grow on selective media (e.g., ura (uracil), leu (leucine), trp (tryptophan), his (histidine)). Usually, plasmids contain antibiotic resistance marker to force the bacterial cell to maintain the plasmid. In an embodiment of the invention, the DNA construct may comprise a nucleotide sequence of a kanamycin resistance marker. In a specific embodiment of the invention, the nucleotide sequence for conferring kanamycin resistance comprises the sequence of SEQ ID 12 (and wherein the sequence is referred to as “KanR” in FIGS. 1 and 3):

ATGAGCCATATTCAACGGGAAACGTCTTGCTCTAGGCCGCGATTAAA
TTCCAACATGGATGCTGATTTATATGGGTATAAATGGGCTCGCGATA
ATGTCGGGCAATCAGGTGCGACAATCTATCGATTGTATGGGAAGCCC
GATGCGCCAGAGTTGTTTCTGAAACATGGCAAAGGTAGCGTTGCCAA
TGATGTTACAGATGAGATGGTCAGACTAAACTGGCTGACGGAATTTA
TGCCTCTTCCGACCATCAAGCATTTTATCCGTACTCCTGATGATGCA
TGGTTACTCACCACTGCGATCCCCGGGAAAACAGCATTCCAGGTATT
AGAAGAATATCCTGATTCAGGTGAAAATATTGTTGATGCGCTGGCAG
TGTTCCTGCGCCGGTTGCATTCGATTCCTGTTTGTAATTGTCCTTTT
AACAGCGATCGCGTATTTCGTCTCGCTCAGGCGCAATCACGAATGAA
TAACGGTTTGGTTGATGCGAGTGATTTTGATGACGAGCGTAATGGCT
GGCCTGTTGAACAAGTCTGGAAAGAAATGCATAAACTTTTGCCATTC
TCACCGGATTCAGTCGTCACTCATGGTGATTTCTCACTTGATAACCT
TATTTTTGACGAGGGGAAATTAATAGGTTGTATTGATGTTGGACGAG
TCGGAATCGCAGACCGATACCAGGATCTTGCCATCCTATGGAACTGC
CTCGGTGAGTTTTCTCCTTCATTACAGAAACGGCTTTTTCAAAAATA
TGGTATTGATAATCCTGATATGAATAAATTGCAGTTTCATTTGATGC
TCGATGAGTTTTTCTAA.

The DNA construct may comprise a nucleotide sequence encoding a promoter operably linked to the nucleic acid sequence encoding the antibiotic resistance marker. Such a promotor may increase the expression of the antibiotic resistance markers discussed in the previous paragraph. In an embodiment of the invention, the promoter for ampicillin resistance is an AmpR promoter which is not only capable of promoting expression of ampicillin resistance markers but also capable of promoting expression of kanamycin resistance markers. In a specific embodiment of the invention, the nucleic acid sequence of the AmpR promoter comprises the sequence of SEQ ID 13 (and wherein the sequence is referred to as “AmpR promoter” in FIGS. 1 and 3):

CGCGGAACCCCTATTTGTTTATTTTTCTAAATACATTCAAATATGTA
TCCGCTCATGAGACAATAACCCTGATAAATGCTTCAATAATATTGAA
AAAGGAAGAGT

The DNA construct may comprise a nucleotide sequence encoding for both of the rrnB T1 terminator and the rrnB T2 terminator. The rrnB T1 and T2 terminators are both efficient transcription terminators in isolated forms, however, when used together, rrnB T1 and T2 terminators may more efficiently terminate transcription.

In an embodiment, the nucleotide sequence of the rrnB T1 terminator comprises the sequence of SEQ ID 14:

CAAATAAAACGAAAGGCTCAGTCGAAAGACTGGGCCTTTCGTTTTAT
CTGTTGTTTGTCGGTGAACGCTCTCCTGAGTAGGACAAAT

In an embodiment, the nucleotide sequence of the rrnB T2 terminator comprises the sequence of SEQ ID 15:

AGAAGGCCATCCTGACGGATGGCCTTTT

The DNA construct may further comprise an origin of replication which is a particular nucleotide sequence at which DNA replication is initiated. DNA replication may proceed from this point bidirectionally or unidirectionally. Some commonly used origins of replication are ColE1, pMB1, pSC101, R6K, pBR322, R6K, p15A, and pUC. In an embodiment of the invention, the origin of replication is pMB1 or derivatives thereof. In a specific embodiment of the invention, the nucleic acid sequence of pMB1 comprises the sequence of SEQ ID 16:

TTTCCATAGGCTCCGCCCCCCTGACGAGCATCACAAAAATCGACGCT
CAAGTCAGAGGTGGCGAAACCCGACAGGACTATAAAGATACCAGGCG
TTTCCCCCTGGAAGCTCCCTCGTGCGCTCTCCTGTTCCGACCCTGCC
GCTTACCGGATACCTGTCCGCCTTTCTCCCTTCGGGAAGCGTGGCGC
TTTCTCATAGCTCACGCTGTAGGTATCTCAGTTCGGTGTAGGTCGTT
CGCTCCAAGCTGGGCTGTGTGCACGAACCCCCCGTTCAGCCCGACCG
CTGCGCCTTATCCGGTAACTATCGTCTTGAGTCCAACCCGGTAAGAC
ACGACTTATCGCCACTGGCAGCAGCCACTGGTAACAGGATTAGCAGA
GCGAGGTATGTAGGCGGTGCTACAGAGTTCTTGAAGTGGTGGCCTAA
CTACGGCTACACTAGAAGGACAGTATTTGGTATCTGCGCTCTGCTGA
AGCCAGTTACCTTCGGAAAAAGAGTTGGTAGCTCTTGATCCGGCAAA
CAAACCACCGCTGGTAGCGGTGGTTTTTTTGTTTGCAAGCAGCAGAT
TACGCGCAGAAAAAAAGGATCTCAA

In an embodiment, the DNA construct is an expression vector which comprises a nucleotide sequence encoding one or more of:
rhaBAD promoter,
RhaR transcription activator,
RhaS transcription activator,
an antibiotic resistance marker,
a promoter operably linked to the nucleic acid encoding the antibiotic resistance marker,
at least one terminator, and
an origin of replication.

In an embodiment, the expression vector comprises a nucleotide sequence encoding:
rhaBAD promoter,
RhaR transcription activator,
RhaS transcription activator,
an antibiotic resistance marker,
a promoter operably linked to the nucleic acid sequence encoding the antibiotic resistance marker,
rrnB T1 terminator,
rrnB T2 terminator, and
an origin of replication.

In an embodiment, the expression vector comprises a nucleotide sequence encoding:
rhaBAD promoter,
RhaR transcription activator,
RhaS transcription activator,
a kanamycin resistance marker,
AmpR promoter,
rrnB T1 terminator,
rrnB T2 terminator, and
pMB1 origin of replication.

In an embodiment, the expression vector comprises nucleotide sequences encoding:
rhaBAD promoter comprising the nucleotide sequence of SEQ ID 8,
RhaR transcription activator comprising the nucleotide sequence of SEQ ID 9,
RhaS transcription activator comprising the nucleotide sequence of SEQ ID 11,
a kanamycin resistance marker comprising the nucleotide sequence of SEQ ID 12,
AmpR promoter comprising the nucleotide sequence of SEQ ID 13,
rrnB T1 terminator comprising the nucleotide sequence of SEQ ID 14,
rrnB T2 terminator comprising the nucleotide sequence of SEQ ID 15, and
pMB1 origin of replication comprising the nucleotide sequence of SEQ ID 16.

The nucleotide sequence encoding a recombinant protein which is to be cloned into the DNA constructs described above may comprise a nucleic acid encoding a monoclonal antibody or fragment thereof, preferably Certolizumab. The nucleic acid encoding Certolizumab comprises a nucleic acid encoding the light and heavy chains of Certolizumab.

In an embodiment, the nucleotide sequence encoding the light chain of Certolizumab comprises the sequence of SEQ ID 5:

gatattcagatgactcagagcccaagttcgctgagcgcttctgttgg
cgatcgtgtgaccattacatgcaaagcctcacagaacgttggtacca
atgtcgcctggtatcagcagaaacctggaaaagcgcccaaagcgctc
atctactcagcgagcttcctgtattcaggcgtgccgtatcgctttag
cggctctggttccggtacagactttaccctcacgatttcgtccttac
aaccggaagatttcgccacgtactattgccagcaatacaacatctat
ccgctgacctttggacaaggcaccaaagtggagatcaaacgcactgt
tgctgcaccgagtgtgttcatctttccaccgtctgatgagcagctga
agtctggtacagcaagtgttgtgtgtctgctgaacaacttctatccg
cgtgaagctaaagtacagtggaaagtcgacaatgccttgcaatccgg
gaatagccaggaaagcgtgactgaacaggacagcaaggattcgacct
acagtctgagcagtaccttaaccttgtcgaaagcggattacgagaaa
cacaaggtctatgcctgtgaagtcacgcaTCAAGGCCTGTCATCGCC
TGTTACTAAATCATTTAATAGAGGAGAATGTTAA

In an embodiment of the invention, the nucleotide sequence encoding the heavy chain of Certolizumab comprises the sequence of SEQ ID 6:

gaagtgcagcttgtggagtctggaggtggcttagtccagccaggtgg
ttccctgcgcttgtcctgtgcagcgagcgggtatgtAttcacagatt
atggcatgaactgggttcggcaagcaccaggcaaaggcctcgaatgg
atggggtggatcaacacgtatattggggaaccgatttatgcggatag
cgtcaaaggtcgcttcacgttcagtctggataccagcaaatcaaccg
cgtatctccagatgaatagcctccgtgctgaagatactgccgtgtac
tactgtgcgcgtggttatcgcagttatgcgatggattactggggcca
aggcaccttagtcaccgttagttctgcctccaccaaaggcccatcag
tgtttccgctggccccttcgtctaaatcgacgagtggtggcacagcc
gcactgggatgcctggtcaaagactactttcccgaacctgtaaccgt
aagctggaatagtggtgctttgacctcaggcgtgcatacgtttccgg
ctgtcctgcagtcatccggtctgtactcgctttcgagcgttgttact
gtaccctctagctccctgggcacccagacgtacatctgcaatgtgaa
ccataagccgtcgaacaccaaagtggacaagaaagttgagccgaaaa
gctgcgacaaaacgcacacatgtgccgccTAA

In an embodiment, the nucleic acid encoding the light and heavy chains of Certolizumab further comprises a nucleotide sequence encoding a signal peptide operably linked to either one or both of the nucleotide sequences encoding the heavy and light chains of Certolizumab. The signal peptide is preferably selected from the group consisting of MalE, OmpA, PhoA, DsbA and Pelb. The nucleic acid sequence encoding the signal peptide is preferably a nucleotide sequence encoding the PelB signal peptide.

In an embodiment, the nucleotide sequence encoding the PelB signal peptide which is operably linked to the nucleotide sequences encoding the light chain of Certolizumab comprises the sequence of SEQ ID 18 and is in the present invention also referred to as PelB signal sequence 1 (see FIG. 3) and abbreviated PelB1:

ATGAAGTATCTtCTGCCGACCGCAGCAGCGGGTCTGCTGCTGCTGGC
AGCACAGCCTGCAATGGCA

In an embodiment of the invention, the nucleotide sequence encoding the PelB signal peptide which is operably linked to the nucleotide sequences encoding the heavy chain of Certolizumab comprises the sequence of SEQ ID 19 and is in the present invention also referred to as PelB signal sequence 2 (see FIG. 3) and abbreviated PelB2:

ATGAAGTATCTGTTGCCGACTGCTGCAGCGGGACTGCTGCTGTTAGC
GGCACAACCGGCGATGGCG

In an embodiment, the DNA constructs comprises an improved TIR of SEQ ID 20:

TTGCTCATGAAGTAT

In an embodiment, the DNA constructs comprises an improved TIR of SEQ ID 21:

TGTTAAATGAAGTAT

In an embodiment, the DNA construct comprises TIRs of nucleotide sequence SEQ ID No 20 and 21 and wherein these TIR nucleotide sequences will comprises at least the first 9 nucleotides of a signal peptide nucleotide sequence of SEQ ID 18 and 19, respectively.

In a specific embodiment of the invention, DNA construct comprises the nucleotide sequence of SEQ ID 17 which is also referred to as KTXHIS-Cert-PelB1-LC-PelB2-HC in the present invention. This DNA construct is preferably an expression vector.

The bacterial host cell to be used for producing the recombinant protein comprises a chromosome having a mutation or modification which disables rhamnose metabolism. The bacterial host cell may be an E. coli cell. In a preferred embodiment, the bacterial host cell is an E. coli K-12 cell, more preferably the bacterial host cell is an E. coli W3110 cell. The disabled rhamnose metabolism is achieved by a mutation in the nucleotide sequence encoding RhaB which renders RhaB inactive. Alternatively, the disabled rhamnose metabolism is achieved by using a bacterial host cell having a chromosome in which the nucleotide sequence encoding RhaB is deleted; this can e.g. be achieved by deleting the nucleotide sequence encoding RhaB. Preferably, the chromosome of the bacterial host cell comprises the nucleic acid sequence encoding RhaT, i.e. the RhaT gene is intact.

In a specific embodiment of the invention, the bacterial host cell is E. coli W3110 rhaBfs ΔDegP Δprc sprW148R.

In a specific embodiment of the invention, the bacterial host cell E. coli W3110 rhaBfs ΔDegP Δprc sprW148R comprises the KTXHIS-Cert-PelB1-LC-PelB2-HC expression vector and is used in a method of expressing Certolizumab which can be used in the production of a Certolizumab biosimilar.

A biosimilar is a highly similar to the reference product, i.e. a Certolizumab biosimilar has highly similar molecular structure and function (i.e. bioactivity) as Certolizumab pegol (Cimzia®) which is produced by the reference product sponsor (i.e. the originator). Moreover, a biosimilar has no clinically meaningful differences from a reference product and the clinical trials that are conducted on biosimilars assess pharmacokinetics and immunogenicity. Most importantly, biosimilars: a) meet medical agency standards of approval, (b) are manufactured in medical agency licensed facilities, and (c) are tracked as part of post-market surveillance to ensure continued safety (as indicated in https://www.fda.gov/media/108905/download).

The present invention can be exemplified as disclosed in the examples 1-9 in the below non-limiting EXAMPLES section.

It should be understood that these examples, relating to the XB166 host cell and the KTXHIS-Cert-PelB1-LC-PelB2-HC expression vector, as well as their combined use, while indicating preferred embodiments of the invention, are given by way of illustration only. From the above disclosed embodiments of the invention and the following examples, one skilled in the art can ascertain the essential characteristics of this invention, and without departing from the spirit and scope thereof, can make various changes and modifications of the invention to adapt it to various types of therapeutic antibodies and immunoglobulins. Thus, various modifications of the invention in addition to those shown and described herein will be apparent to those skilled in the art from the foregoing description. Such modifications are also intended to fall within the scope of the appended claims. An example of such a modification is that one or more of the above indicated SEQ IDs 1-21 may be replaced by a sequence with at least 90% sequence identity thereto.

### EXAMPLES

Example 1 relates to the construction of the XB166 host cell which is in the present invention also referred to as E. coli W3110 rhaBfs ΔDegP Δprc sprW148R.

Example 2 relates to the construction of the KTXHIS-Cert-PelB1-LC-PelB2-HC expression vector.

Example 3 relates to the light and the heavy chains of Certolizumab expressed by the expression vector in disclosed in Example 2.

Example 4 shows that Certolizumab produced according to the present invention is free of bacteriophages.

Example 5 relates to TIR library selection and the isolation of evolved TIRs.

Example 6 relates to Western-blot analysis of media fraction expression—the results show that the E83 expression vector which comprised the synthetically evolved TIR for the regulation of Certolizumab heavy chain expression resulted in the highest levels of Certolizumab.

Example 7 relates to Western-blot analysis of total fraction expression—the results show that the E83 expression vector which comprised the synthetically evolved TIR for the regulation of Certolizumab heavy chain expression resulted in the highest levels of Certolizumab.

Example 8 relates to NanoDrop® and ÄKTA chromatography which was performed to detect yield and titer differences between expression systems XB62 (XB17 host cell containing D37 expression vector having un-evolved TIRs and a wild-type nucleotide sequence for the PelB signal peptide upstream of each of the nucleotide sequences for the light and heavy chains of Certolizumab) and XB102 (XB17 host cell containing expression vector E83 having synthetically engineered TIR for the regulation of the Certolizumab heavy chain expression)—the results show that the expression system XB102 which comprises the expression vector E83 containing the synthetically engineered TIR (for the regulation of the Certolizumab heavy chain expression) results in highest yields and titer of Certolizumab.

Example 9 relates to the comparison of Certolizumab expression in host cells XB17 and XB166—the results show that the use of the XB166 host cell (E. coli W3110 rhaBfs ΔDegP Δprc sprW148R) in combination with E111 (KTXHIS-Cert-Pelb1-LC-Pelb2-HC) results in highest relative yields of Certolizumab.

### Example 1—Strain Development

The XB166 host cell is an E. coli W3310 derivative which was genetically engineered for the production of recombinant proteins such as antibodies and antibody fragments. Moreover, the XB166 host cell was designed as a strain for a rhamnose inducible system where the nucleotide sequence encoding the recombinant protein of interest is cloned into the KTXHIS plasmid and expressed under the control of a rhamnose inducible promoter.

The XB166 host cell was developed from the parental E. coli strain W3110 which was obtained from the E. coli Genetic Stock Center (CGSC), Yale University (New Haven, USA), Catalog No.: 4474. Genotype: F-, λ-, IN(rrnD-rrnE)1, rph-1.

XB166 was customized for efficient induction from a rhamnose-dependent promoter by deactivating the rhab gene [7]. Moreover, three genomic modifications useful for the production of antibody fragments [8], were introduced as described below subsections in detail.

In summary, the method for developing the XB166 host cell involved the following listed modifications which will be discussed in detail in the below subsections:
a) RhaB frame shift mutation;
b) degP deletion;
c) prc deletion; and
d) sprW148R mutation.
   Step (a)—Construction of the RhaB Frame Shift Mutant.

The parental E. coli strain W3110 was engineered to generate a derivative with a frameshift in the chromosomal copy of rhaB making it unable to utilize rhamnose as a carbon source. To this end, cells were genetically engineered using the gene replacement plasmid pMAK705-rhaBfs [9].

The engineered strain was phenotypically tested to verify that rhamnose cannot be utilized as carbon source anymore. Furthermore, the chromosomal fragment containing the rhaB frameshift was PCR amplified and the PCR product was sequenced to confirm the correct insertion of two bases (see underlined CG bases in below disclosed SEQ ID NO 22):

tgtggcagcaactgattcagcccggcgagaaactgaaatcgatccgg
cgagcgatacagcacattggtcagacacagattatcggtatgttcat
acagatgccgatcatgatcgcgtacgaaacagaccgtgccaccggtg
atggtatagggctgcccattaaacacatgaatacccgtgccatgttc
gacaatcacaatttcatgaaaatcatgatgatgttcaggaaaatccg
cctgcgggagccggggttctatcgccacggacgcgttaccagacgga
aaaaaatccacactatgtaatacggtcatactggcctcctgatgtcg
tcaacacggcgaaatagtaatcacgaggtcaggttcttaccttaaat
tttcgacggaaaaccacgtaaaaaacgtcgatttttcaagatacagc
gtgaattttcaggaaatgcggtgagcatcacatcaccacaattcagc
aaattgtgaacatcatcacgttcatctttccctggttgccaatggcc
cattttcctgtcagtaacgagaaggtcgcgaattcaggcgcttttta
gactggtcgtaatgaaattcagcaggatcacattatgacctttcgca
attgtgtcgccgtcgatctcggcgcatccagtgggcgcgtgatgctg
gcgcgttacgagcgtgaatgccgcagcctgacgctgcgcgaaatcca
tcgttttaacaatgggctgcatagtcagaacggctatgtcacctggg
atgtggatagcctGgaaagtgccattcgccttggattaaacaaggtg
tgcgaggaagggattcgtatcgCGatagcattgggattgatacctgg
ggcgtggactttgtgctgctcgaccaacagggtcagcgtgtgggcct
gcccgttgcttatcgcgatagccgcaccaatggcctaatggcgcagg
cacaacaacaactcggcaaacgcgatatttatcaacgtagcggcatc
cagtttctgcccttcaatacgctttatcagttgcgtgcgctgacgga
gcaacaacctgaacttattccacacattgctcacgctctgctgatgc
cggattacttcagttatcgcctgaccggcaagatgaactgggaatat
accaacgccacgaccacgcaactggtcaatatcaatagcgacgactg
ggacgagtcgctactggcgtggagcggggccaacaaagcctggtttg
gtcgcccgacgcatccgggtaatgtcataggtcactggatttgcccg
cagggtaatgagattccagtggtcgccgttgccagccatgataccgc
cagcgcggttatcgcctcgccgttaaacggctcacgtgctgcttatc
tctcttctggcacctggtcattgatgggcttcgaaagccagacgcca
tttaccaatgacacggcactggcagccaacatcaccaatgaaggcgg
ggcggaaggtcgctatcgggtgctgaaaaatattatgggcttatggc
tgcttcagcgagtgcttcaggagcagcaaatcaacgatcttccggcg
cttatctccgcgacacaggcacttccggcttgccgcttcattatcaa
tcccaatgacgatcgct

A single colony was picked and grown in LB vegitone to prepare a glycerol stock. The resulting strain E. coli W3110 rhaBfs which was designated as XB17 served as starting strain for the following genetic modifications.

Step (b)—degP Deletion

The XB17 (E. coli W3110 rhaBfs) strain was further engineered to be equipped with three key modifications (degP prc spr) in order to create a “triple-mutant” host strain allowing high-level accumulation of recombinant antibody fragments due to reduced proteolytic degradation of the light chain in the periplasm [8].

To this end, the genomic copy of the gene coding for the periplasmic serine endoprotease DegP was knocked-out using the gene replacement plasmid pMAK705-sacB-DegP, in which a fragment homologous to the degP upstream region is fused to a fragment homologous to the degP downstream region. In order to avoid polar effects, the gene replacement cassette was designed to preserve the degP start codon as well as the last 7 degP codons. Next to a temperature-sensitive origin of replication, this gene replacement plasmid also carries the sacB gene for counterselection, thus facilitating plasmid curing after strain construction.

Deletion of the chromosomal degP gene was confirmed by PCR amplification of the degP “scar” region and sequencing of the PCR product (see below SEQ ID 23 wherein the underlined codon is the degP start codon and the codons in bold letters are the last 7 codons of the degP gene):

atataaaaatgtcgctgtaaaacatgtgtttagccatccagatgtcgagcggcttgaattgcagggcta
tcgggtcattagcggattattagagatttatcgtcctttattaagcctgtcgttatcagactttactga
actggtagaaaaagaacgggtgaaacgtttccctattgaatcgcgcttattccacaaactctcgacgcg
ccatcggctggcctatgtcgaggctgtcagtaaattaccgtcagattctcctgagtttccgctatggga
atattattaccgttgccgcctgctgcaggattatatcagcggtatgaccgacctctatgcgtgggatga
ataccgacgtctgatggccgtagaacaataaccaggcttttgtaaagacgaacaataaatttttacctt
ttgcagaaactttagttcggaacttcaggctataaaacgaatctgaagaacacagcaattttgcgttat
ctgttaatcgagactgaaatacATGatctacctgttaatgcagTAAtctccctcaaccccttcctgaaa
acgggaaggggttctccttacaatctgtgaacttcaccacaactccatacatcttcatcatcctttagg
catttgcacaatgccgtacgttacgtacttccttatgctaagccgtgcataacggaggacttatggctg
gctggcatcttgataccaaaatggcgcaggatatcgtggcacgtaccatgcgcatcatcgataccaata
tcaacgtaatggatgcccgtgggcgaattatcggcagcggcgatcgtgagcgtattggtgaattgcacg
aaggtgcattgctggtactttcacagggacgagtcgtcgatatcgatgacgcggtagcacgtcatctgc
acggtgtgcggcaggggattaatctaccgttacggctggaaggtgaaattgtcggcgtaattggcctga
caggtgaaccagagaatctgcgtaaatatggcgaactggtctgcatgacggc

A single colony of the engineered strain was picked and grown in LB vegitone to prepare a glycerol stock. The resulting strain E. coli W3110 rhaBfs ΔDegP which was designated as XB83 served as starting strain for the following genetic modifications.

Step (c)—Prc Deletion

With E. coli W3110 rhaBfs ΔDegP (XB83) as starting strain, the prc gene was knocked-out using the gene replacement plasmid pMAK705-sacB-prc in analogy to the above described degP deletion. Deletion of the chromosomal prc gene was confirmed by PCR amplification of the prc scar region and sequencing of the PCR product (see below SEQ ID 24 wherein the underlined codon is the prc start codon and the codons in bold letters are the last 7 codons of the prc gene):

tttacggtgttaaacccggcgcaacgcgtgtcgatcttgacggcaacccatgcggtgagctggacgagc
aacatgtagagcatgctcgcaagcagcttgaagaagcgaaagcgcgtgttcaggcacagcgtgctgaac
agcaagcgaaaaaacgcgaagctgccgcaactgctggtgagaaagaagacgcaccgcgccgcgaacgca
agccacgtccgactacgccacgccgcaaagaaggcgctgaacgtaaacctcgtgcgcaaaagccggtag
agaaagcgccaaaaacagtaaaagcacctcgcgaagaacagcacaccccggtttctgacatttcagctc
tgactgtcggacaagccctgaaggtgaaagcgggtcaaaacgcgatggatgccaccgtattagaaatca
ccaaagacggcgtccgcgtccagctgaattcgggtatgtctttgattgtgcgcgcagaacacctggtgt
tctgaaacggaggccgggccaggcATGcaacccgctcccgtcaagTAAtatcaatcaggcacaagaaat
tgtgcctgattttttaacagcgacaagatgccgtaaatcagatgctacaaaatgtaaagttgtgtcttt
ctggtgacttacgcactatccagacttgaaaatagtcgcgtaacccatacgatgtgggtatcgcatatt
gcgttttgttaaactgaggtaaaaagaaaattatgatgcgaatcgcgctcttcctgctaacgaacctgg
ccgtaatggtcgttttcgggctggtactgagcctgacagggatacagtcgagcagcgttcaggggctga
tgatcatggccttgctgttcggttttggtggttccttcgtttcgcttctgatgtccaaatggatggcat
tacgatctgttggcggggaagtgatcgagcaaccgcgtaacgaaagggaacgttggctggtcaatactg
tagcaacccaggctcgtcaggggggatcgctatgccgcaagtggctatctacc

A single colony of the engineered strain was picked and grown in LB vegitone to prepare a glycerol stock. The resulting strain E. coli W3110 rhaBfs ΔdegP Δprc designated here as XB152 served as starting strain for final genetic engineering step.

Step (d)—sprW148R

The final step of engineering the expression host was devoted to complement the triple mutant genotype by the introduction of the sprW148R mutation leading to an amino acid substitution in the spr gene. While the deletion of degP and prc is expected to result in a strain with reduced proteolytic degradation of the light chain of the antibody fragments, the spr mutation is described to produce higher amounts of recombinant protein [8].

Using E. coli W3110 rhaBfs ΔDegP Δprc (XB152) as starting strain, the genomic copy of the spr gene was engineered using the gene replacement plasmid pMAK705-sacB-sprW148R containing the mutant spr fragment [10]. The chromosomal fragment containing the sprW148R mutation was PCR amplified and the PCR product was sequenced to confirm that the genomic spr gene was correctly replaced by the mutant allele (see below SEQ ID 25; the T to A mutation is highlighted in bold):

aacaaacaacatggtcaaatctcaaccgattttgagatatatcttgcgcgggattcccgcgattgcagt
agcggttctgctttctgcatgtagtgcaaataacaccgcaaagaatatgcatcctgagacacgtgcagt
gggtagtgaaacatcatcactgcaagcttctcaggatgaatttgaaaacctggttcgtaatgtcgacgt
aaaatcgcgaattatggatcagtatgctgactggaaaggcgtacgttatcgtctgggcggcagcactaa
aaaaggtatcgattgttctggtttcgtacagcgtacattccgtgagcaatttggcttagaacttccgcg
ttcgacttacgaacagcaggaaatgggtaaatctgtttcccgcagtaatttgcgtacgggtgatttagt
tctgttccgtgccggttcaacgggacgccatgtcggtatttatatcggcaacaatcagtttgtccatgc
ttccaccagcagtggtgttattatttccagcatgaatgaaccgtacAggaagaagcgttacaacgaagc
acgccgggttctcagccgcagctaataaaccgtttggatgcaatcccttggctatcctgacgagttaac
tgaaagcactgcttaggcagtgcttttttgttttcattcatcagagaaaatgatgtttccgcgtcttga
tccaggctatagtccggtcattgttatcttttaaatgttgtcgtaatttcaggaaattaacggaatcat
gttcatacgcgctcccaattttggacgtaagctcctgcttacctgcattgttgcaggcgtaatgattgc
gatactggtgagttgccttcagtttttagtggcctggcataagcacgaagtcaaatacgacacactgat
taccgacgtacaaaagtatctcgatacctattttgccgacctgaaatccactactgaccggctccagcc
gctgaccttagatacctgccagcaagctaaccccgaactgaccgcccgcgcagcgtttagcatgaatgt
ccgaacgtttgtgctggtgaaagataaaa

A single colony of the engineered strain was picked and grown in LB vegitone to prepare a glycerol stock. The resulting strain E. coli W3110 rhaBfs ΔDegP Δprc sprW148R was designated as XB166.

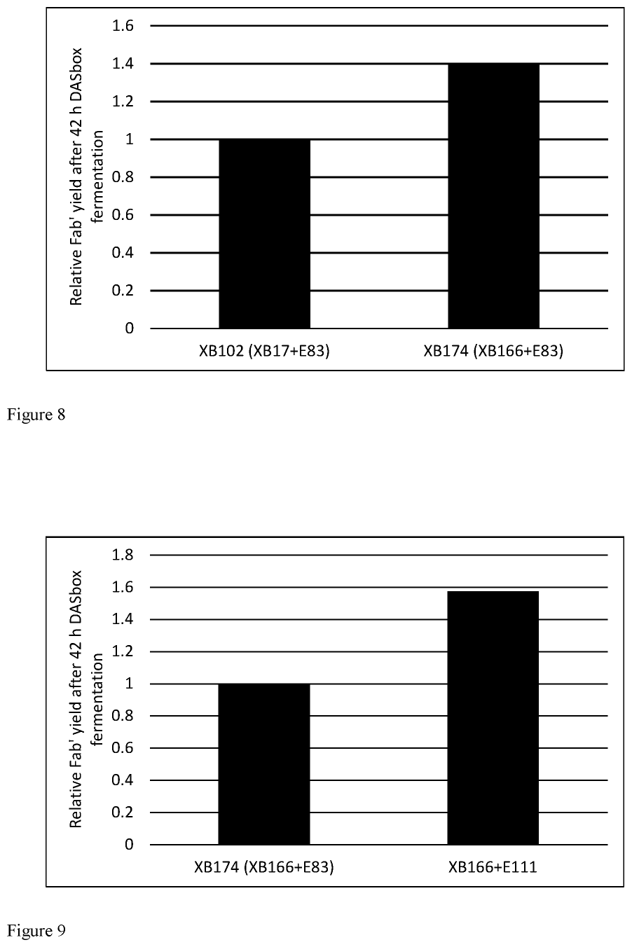

The advantageous technical effects of the XB166 is shown and discussed in detail in Example 8 and FIG. 8.

### Example 2—KTXHIS-Cert-PelB1-LC-PelB2-HC

The first construct of Certolizumab that was made was with signal peptides OmpA-LC, PelB-HC (gene synthesized) and cloned into the KTXHIS plasmid via EcoRI and HindIII sites. From this first construct, the signal peptide(s) was then exchanged with the use of homologous recombination of PCR fragments in E coli. I've made several versions of PelBLC, PelBHC in KTXHIS vector where, even though the resulting amino acid sequence of PelB would be the same, the codons were different. I'm not sure which one was used as the starting plasmid by Kiavash to be honest.

The mother construct for the expression of Certolizumab was made was with signal peptides OmpA (upstream of nucleotide sequence encoding light chain) and PelB (upstream of nucleotide sequence encoding the heavy chain) and cloned into the KTXHIS plasmid (see Seq ID 1) via EcoRI and HindIII sites (see SEQ ID 2). From this mother construct, the existing signal peptide nucleotide sequences were then swapped with signal peptide nucleotide sequences of SEQ ID 18 and 19 by using homologous recombination of PCR fragments in E. coli yielding expression vector D37. Post synthetic construction of both TIRs regulating the expression of light and heavy chains of Certolizumab (as explained in the below Example 5-10), an expression vector comprising SEQ ID 17 was generated.

The expression plasmid KTXHIS has previously been described in European patent application EP20201096.3. The resulting nucleotide sequence comprises a nucleotide sequence of SEQ ID 17 and is referred to as KTXHIS-Cert-PelB1-LC-PelB2-HC in the present invention. The plasmid map of KTXHIS-Cert-PelB1-LC-PelB2-HC is illustrated in FIG. 3:

GGCTGTTTTGGCGGATGAGAGAAGATTTTCAGCCTGATACAGATTAA
ATCAGAACGCAGAAGCGGTCTGATAAAACAGAATTTGCCTGGCGGCA
GTAGCGCGGTGGTCCCACCTGACCCCATGCCGAACTCAGAAGTGAAA
CGCCGTAGCGCCGATGGTAGTGTGGGGTCTCCCCATGCGAGAGTAGG
GAACTGCCAGGCATCAAATAAAACGAAAGGCTCAGTCGAAAGACTGG
GCCTTTCGTTTTATCTGTTGTTTGTCGGTGAACGCTCTCCTGAGTAG
GACAAATCCGCCGGGAGCGGATTTGAACGTTGCGAAGCAACGGCCCG
GAGGGTGGCGGGCAGGACGCCCGCCATAAACTGCCAGGCATCAAATT
AAGCAGAAGGCCATCCTGACGGATGGCCTTTTTGCGTTTCTACAAAC
TCTTTTGTTTATTTTTCTAAATACATTCAAATATGTATCCGCTCATG
AGACTAGGCTTCCGCGCCCTCATCCGAAAGGGCGTATTCATATATGC
GGTGTtAAATACCGCACAGATGCGTAAGGAGAAAATACCGCATCAGG
CGCTCTTCCGCTTCCTCGCTCACTGACTCGCTGCGCTCGGTCGTTCG
GCTGCGGCGAGCGGTATCAGCTCACTCAAAGGCGGTAATACGGTTAT
CCACAGAATCAGGGGATAACGCAGGAAAGAACATGTGAGCAAAAGGC
CAGCAAAAGGCCAGGAACCGTAAAAAGGCCGCGTTGCTGGCGTTTTT
CCATAGGCTCCGCCCCCCTGACGAGCATCACAAAAATCGACGCTCAA
GTCAGAGGTGGCGAAACCCGACAGGACTATAAAGATACCAGGCGTTT
CCCCCTGGAAGCTCCCTCGTGCGCTCTCCTGTTCCGACCCTGCCGCT
TACCGGATACCTGTCCGCCTTTCTCCCTTCGGGAAGCGTGGCGCTTT
CTCATAGCTCACGCTGTAGGTATCTCAGTTCGGTGTAGGTCGTTCGC
TCCAAGCTGGGCTGTGTGCACGAACCCCCCGTTCAGCCCGACCGCTG
CGCCTTATCCGGTAACTATCGTCTTGAGTCCAACCCGGTAAGACACG
ACTTATCGCCACTGGCAGCAGCCACTGGTAACAGGATTAGCAGAGCG
AGGTATGTAGGCGGTGCTACAGAGTTCTTGAAGTGGTGGCCTAACTA
CGGCTACACTAGAAGGACAGTATTTGGTATCTGCGCTCTGCTGAAGC
CAGTTACCTTCGGAAAAAGAGTTGGTAGCTCTTGATCCGGCAAACAA
ACCACCGCTGGTAGCGGTGGTTTTTTTGTTTGCAAGCAGCAGATTAC
GCGCAGAAAAAAAGGATCTCAAGAAGATCCTTTGATCTTTTCTACGG
GGTCTGACGCTCAGTGGAACGAAAACTCACGTTAAGGGATTTTGGTC
ATGAGATTATCAAAAAGGATCTTCACCTAGATCCTTTTAAATTAAAA
ATGAAGTTTTAAATCAATCTAAAGTATATATGAGTAAACTTGGTCTG
ACAGTTAGAAAAACTCATCGAGCATCAAATGAAACTGCAATTTATTC
ATATCAGGATTATCAATACCATATTTTTGAAAAAGCCGTTTCTGTAA
TGAAGGAGAAAACTCACCGAGGCAGTTCCATAGGATGGCAAGATCCT
GGTATCGGTCTGCGATTCCGACTCGTCCAACATCAATACAACCTATT
AATTTCCCCTCGTCAAAAATAAGGTTATCAAGTGAGAAATCACCATG
AGTGACGACTGAATCCGGTGAGAATGGCAAAAGTTTATGCATTTCTT
TCCAGACTTGTTCAACAGGCCAGCCATTACGCTCGTCATCAAAATCA
CTCGCATCAACCAAACCGTTATTCATTCGTGATTGCGCCTGAGCGAG
ACGAAATACGCGATCGCTGTTAAAAGGACAATTACAAACAGGAATCG
AATGCAACCGGCGCAGGAACACTGCCAGCGCATCAACAATATTTTCA
CCTGAATCAGGATATTCTTCTAATACCTGGAATGCTGTTTTCCCGGG
GATCGCAGTGGTGAGTAACCATGCATCATCAGGAGTACGGATAAAAT
GCTTGATGGTCGGAAGAGGCATAAATTCCGTCAGCCAGTTTAGTCTG
ACCATCTCATCTGTAACATCATTGGCAACGCTACCTTTGCCATGTTT
CAGAAACAACTCTGGCGCATCGGGCTTCCCATACAATCGATAGATTG
TCGCACCTGATTGCCCGACATTATCGCGAGCCCATTTATACCCATAT
AAATCAGCATCCATGTTGGAATTTAATCGCGGCCTAGAGCAAGACGT
TTCCCGTTGAATATGGCTCATACTCTTCCTTTTTCAATATTATTGAA
GCATTTATCAGGGTTATTGTCTCATGAGCGGATACATATTTGAATGT
ATTTAGAAAAATAAACAAATAGGGGTTCCGCGCACATTTCCCCGAAA
AGTGCCACCTGACGTCTAAGAAACCATTATTATCATGACATTAACCT
ATAAAAATAGGCGTATCACGAGGCCCTTTCGTCTATCTTTCTGCGAA
TTGAGATGACGCCACTGGCTGGGCGTCATCCCGGTTTCCCGGGTAAA
CACCACCGAAAAATAGTTACTATCTTCAAAGCCACATTCGGTCGAAA
TATCACTGATTAACAGGCGGCTATGCTGGAGAAGATATTGCGCATGA
CACACTCTGACCTGTCGCAGATATTGATTGATGGTCATTCCAGTCTG
CTGGCGAAATTGCTGACGCAAAACGCGCTCACTGCACGATGCCTCAT
CACAAAATTTATCCAGCGCAAAGGGACTTTTCAGGCTAGCCGCCAGC
CGGGTAATCAGCTTATCCAGCAACGTTTCGCTGGATGTTGGCGGCAA
CGAATCACTGGTGTAACGATGGCGATTCAGCAACATCACCAACTGCC
CGAACAGCAACTCAGCCATTTCGTTAGCAAACGGCACATGCTGACTA
CTTTCATGCTCAAGCTGACCAATAACCTGCCGCGCCTGCGCCATCCC
CATGCTACCTAAGCGCCAGTGTGGTTGCCCTGCGCTGGCGTTAAATC
CCGGAATCGCCCCCTGCCAGTCAAGATTCAGCTTCAGACGCTCCGGG
CAATAAATAATATTCTGCAAAACCAGATCGTTAACGGAAGCGTAGGA
GTGTTTATCATCAGCATGAATGTAAAAGAGATCGCCACGGGTAATGC
GATAAGGGCGATCGTTGAGTACATGCAGGCCATTACCGCGCCAGACA
ATCACCAGCTCACAAAAATCATGTGTATGTTCAGCAAAGACATCTTG
CGGATAACGGTCAGCCACAGCGACTGCCTGCTGGTCGCTGGCAAAAA
AATCATCTTTGAGAAGTTTTAACTGATGCGCCACCGTGGCTACCTCG
GCCAGAGAACGAAGTTGATTATTCGCAATATGGCGTACAAATACGTT
GAGAAGATTCGCGTTATTGCAGAAAGCCATCCCGTCCCTGGCGAATA
TCACGCGGTGACCAGTTAAACTCTCGGCGAAAAAGCGTCGAAAAGTG
GTTACTGTCGCTGAATCCACAGCGATAGGCGATGTCAGTAACGCTGG
CCTCGCTGTGGCGTAGCAGATGTCGGGCTTTCATCAGTCGCAGGCGG
TTCAGGTATCGCTGAGGCGTCAGTCCCGTTTGCTGCTTAAGCTGCCG
ATGTAGCGTACGCAGTGAAAGAGAAAATTGATCCGCCACGGCATCCC
AATTCACCTCATCGGCAAAATGGTCCTCCAGCCAGGCCAGAAGCAAG
TTGAGACGTGATGCGCTGTTTTCCAGGTTCTCCTGCAAACTGCTTTT
ACGCAGCAAGAGCAGTAATTGCATAAACAAGATCTCGCGACTGGCGG
TCGAGGGTAAATCATTTTCCCCTTCCTGCTGTTCCATCTGTGCAACC
AGCTGTCGCACCTGCTGCAATACGCTGTGGTTAACGCGCCAGTGAGA
CGGATACTGCCCATCCAGCTCTTGTGGCAGCAACTGATTCAGCCCGG
CGAGAAACTGAAATCGATCCGGCGAGCGATACAGCACATTGGTCAGA
CACAGATTATCGGTATGTTCATACAGATGCCGATCATGATCGCGTAC
GAAACAGACCGTGCCACCGGTGATGGTATAGGGCTGCCCATTAAACA
CATGAATACCCGTGCCATGTTCGACAATCACAATTTCATGAAAATCA
TGATGATGTTCAGGAAAATCCGCCTGCGGGAGCCGGGGTTCTATCGC
CACGGACGCGTTACCAGACGGAAAAAAATCCACACTATGTAATACGG
TCATACTGGCCTCCTGATGTCGTCAACACGGCGAAATAGTAATCACG
AGGTCAGGTTCTTACCTTAAATTTTCGACGGAAAACCACGTAAAAAA
CGTCGATTTTTCAAGATACAGCGTGAATTTTCAGGAAATGCGGTGAG
CATCACATCACCACAATTCAGCAAATTGTGAACATCATCACGTTCAT
CTTTCCCTGGTTGCCAATGGCCCATTTTCTTGTCAGTAACGAGAAGG
TCGCGAATCCAGGCGCTTTTTAGACTGGTCGTAATGAAATTCAGGAG
GAAtTgctcATGAAGTATCTtCTGCCGACCGCAGCAGCGGGTCTGCT
GCTGCTGGCAGCACAGCCTGCAATGGCAgatattcagatgactcaga
gcccaagttcgctgagcgcttctgttggcgatcgtgtgaccattaca
tgcaaagcctcacagaacgttggtaccaatgtcgcctggtatcagca
gaaacctggaaaagcgcccaaagcgctcatctactcagcgagcttcc
tgtattcaggcgtgccgtatcgctttagcggctctggttccggtaca
gactttaccctcacgatttcgtccttacaaccggaagatttcgccac
gtactattgccagcaatacaacatctatccgctgacctttggacaag
gcaccaaagtggagatcaaacgcactgttgctgcaccgagtgtgttc
atctttccaccgtctgatgagcagctgaagtctggtacagcaagtgt
tgtgtgtctgctgaacaacttctatccgcgtgaagctaaagtacagt
ggaaagtcgacaatgccttgcaatccgggaatagccaggaaagcgtg
actgaacaggacagcaaggattcgacctacagtctgagcagtacctt
aaccttgtcgaaagcggattacgagaaacacaaggtctatgcctgtg
aagtcacgcaTCAAGGCCTGTCATCGCCTGTTACTAAATCATTTAAT
AGAGGAGAATGTTAAATGAAGTATCTGTTGCCGACTGCTGCAGCGGG
ACTGCTGCTGTTAGCGGCACAACCGGCGATGGCGgaagtgcagcttg
tggagtctggaggtggcttagtccagccaggtggttccctgcgcttg
tcctgtgcagcgagcgggtatgtAttcacagattatggcatgaactg
ggttcggcaagcaccaggcaaaggcctcgaatggatggggtggatca
acacgtatattggggaaccgatttatgcggatagcgtcaaaggtcgc
ttcacgttcagtctggataccagcaaatcaaccgcgtatctccagat
gaatagcctccgtgctgaagatactgccgtgtactactgtgcgcgtg
gttatcgcagttatgcgatggattactggggccaaggcaccttagtc
accgttagttctgcctccaccaaaggcccatcagtgtttccgctggc
cccttcgtctaaatcgacgagtggtggcacagccgcactgggatgcc
tggtcaaagactactttcccgaacctgtaaccgtaagctggaatagt
ggtgctttgacctcaggcgtgcatacgtttccggctgtcctgcagtc
atccggtctgtactcgctttcgagcgttgttactgtaccctctagct
ccctgggcacccagacgtacatctgcaatgtgaaccataagccgtcg
aacaccaaagtggacaagaaagttgagccgaaaagctgcgacaaaac
gcacacatgtgccgccTAATAAaagctt

### Example 3—Recombinant Protein—Certolizumab

The amino acid sequence of the light chain of Certolizumab expressed by the expression vector KTXHIS-Cert-PelB1-LC-PelB2-HC comprises the sequence of SEQ ID 3:

DIQMTQSPSSLSASVGDRVTITCKASQNVGTNVAWYQQKPGKAPKAL
IYSASFLYSGVPYRFSGSGSGTDFTLTISSLQPEDFATYYCQQYNIY
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC

The amino acid sequence of the heavy chain of Certolizumab expressed by the expression vector KTXHIS-cert-PelB1-LC-PelB2-HC comprises the sequence of SEQ ID 4:

EVQLVESGGGLVQPGGSLRLSCAASGYVFTDYGMNWVRQAPGKGLEW
MGWINTYIGEPIYADSVKGRFTFSLDTSKSTAYLQMNSLRAEDTAVY
YCARGYRSYAMDYWGQGTLVTVSSASTKGPSVFPLAPSSKSTSGGTA
ALGCLVKDYFPEPVTVSWNSGALTSGVHTFPAVLQSSGLYSLSSVVT
VPSSSLGTQTYICNVNHKPSNTKVDKKVEPKSCDKTHTCAA

Moreover, the resulting PelB signal peptide comprises an amino acid sequence of SEQ ID 3 [6]: MKYLLPTAAAGLLLLAAQPAMA

### Example 4—Cell Banking

The XB166 was transformed with KTXHIS-Cert-Pelb1-LC-Pelb2-HC.

An RCB was prepared by picking a single colony and overnight growth in Defined Bioreactor Medium (DBM). The following day, 20% (final concentration) glycerol was aseptically added, the culture was aliquoted and stored in −80° C.

All materials used in the manufacture of the RCB are of non-animal and non-human origin.

### Example 5—TIR Library Selection and the Isolation of Evolved TIRs

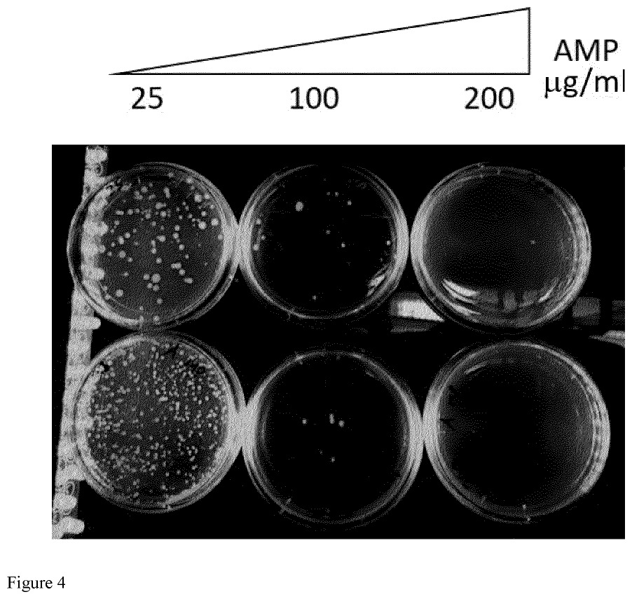

Synthetic evolution was used to select TIRs that were more compatible with the host cell ribosomes by utilizing the methods disclosed in U.S. Pat. No. 10,696,963 and Mirzadeh et al 2015 and briefly described in this example (see also WO21158163 for synthetical evolution of similar TIRs). A TIR library was created with the design that meant the six nucleotides immediately upstream from the ATG start codon were completely randomized, and the six nucleotides immediately downstream from the ATG start codon were randomized with synonymous codon changes only. Each TIR library theoretically contained >18,000 expression plasmids with a different TIR. In this experiment, the expression plasmids comprising the expression cassette PelBss-Certolizumab-LC-PelBss-Certolizumab-HC (wherein “ss” is abbreviation for signal sequence) which in turn was fused to β-lactamase were subjected to the described TIR library generation. For each certolizumab chain in separate experiments, a TIR library was transformed into bacteria and plated onto LB agar plates containing a fixed amount of rhamnose and increasing concentrations of ampicillin (FIG. 4). Colonies from the TIR library plates (upper panel/row in FIG. 4) that were resistant to a high concentration of ampicillin, or were visually bigger than a corresponding colony from the un-evolved expression cassette plates (lower panel/row in FIG. 4), were isolated and the TIRs were sequenced. The identified TIRs (i.e. synthetically evolved TIRs) were then back-engineered into plasmids devoid of the β-lactamase in order to assess their effect on Certolizumab expression in larger fermentation scales.

A specific example of a TIR that resulted by using the above described synthetic evolution method is in the present invention referred to as TIR-LC and has SEQ ID 20 and this TIR-LC regulates the expression of the light chain of Certolizumab. The TIR-LC of SEQ ID 20 is the TIR comprised in the E111 expression vector discussed in Example 10.

### Example 6-Western-Blot Analysis of Media Fraction after 20 Hours of Induced Expression in DASbox® Fermenters (i.e. DASbox® Mini Bioreactor Systems)

Certolizumab was expressed by using:
i. an expression vector referred to as D36 which comprises a codon optimized version of the wild-type PelB signal peptide nucleotide sequence (of SEQ ID 19) upstream of the nucleotide sequence of the heavy chain of Certolizumab, and
ii. expression vectors E81, E82 and E83 which differ from the D36 vector in that the E81, E82 and E83 expression vectors each comprise synthetically engineered modifications of the TIRs responsible for the regulation of the Certolizumab heavy chain expression (i.e. the TIRs upstream of the nucleotide sequence of the Certolizumab heavy chain).

The synthetically engineered TIR (for the regulation of the Certolizumab heavy chain expression) comprised in the E83 expression vector has a nucleotide sequence of SEQ ID 21. The nucleotide sequences of the TIRs upstream of the heavy chains of E81 and E82 (and other expression vectors that were researched, developed and tested) are not shown in the present invention. The aim of the synthetic engineering of the TIRs upstream of the heavy chain nucleotide sequence was to test the effect of nucleotide substitutions on the expression of Certolizumab.

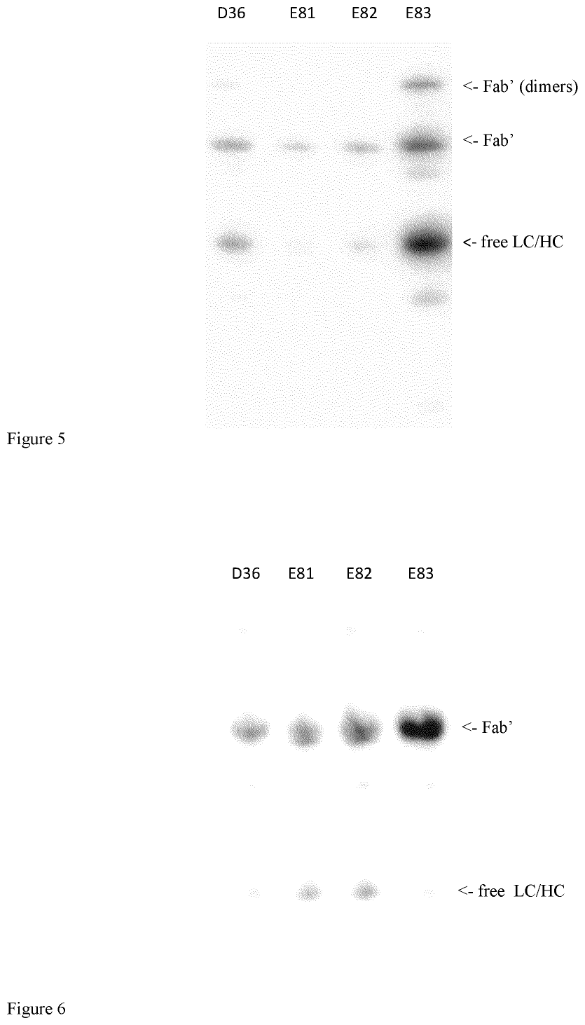

A volume of 100 μl of each Certolizumab containing sample was spun down at 14000×g for 5 minutes after which the supernatant was separated and toped up with H₂O to a total of 100 μl. Next, 100 μl of 2× sample buffer was added to the sample before it was boiled for 5 minutes at 95° C., after which an equal amount of volume was loaded in each well and separated by a 12% SDS-PAGE. Protein levels were visualized by immuno-blotting with antisera to Certolizumab. The results are shown in FIG. 5 which illustrates from left to right: D36, E81, E82 and E83 expression vectors.

As illustrated in the Western-blots in FIG. 5, the E83 expression vector resulted in the highest levels of Certolizumab (indicated as fragment antibody Fab′ in the figure) in the media fraction. There was also signs of Fab′ dimers and free light chain (LC) and heavy chain (HC) present in the sample.

### Example 7—Western-Blot Analysis of Total (Media and Cell) Fraction after 20 Hours of Induced Expression in DASbox® Fermenters

Certolizumab was expressed by using the D36, E81, E82, E83 expression vectors.

A volume of 5 μl of each sample was added to 100 μl of 2× Sample buffer before it was boiled for 5 minutes at 95° C., and an equal amount of volume was loaded in each well and separated by a 12% SDS-PAGE. Protein levels were visualized by immuno-blotting with antisera to Certolizumab. The results are shown in FIG. 6 which illustrates from left to right: D36, E81, E82 and E83 expression vectors.

As illustrated in the Western-blots in FIG. 6, the E83 expression vector (which comprised the synthetically evolved TIR for the regulation of Certolizumab heavy chain expression) resulted in the highest levels of Certolizumab as indicated as fragment antibody Fab′ in the figure).

### Example 8—NanoDrop® and ÄKTA Chromatography

Certolizumab was expressed by using the XB17 host cell (E. coli W3110 rhaBfs) by using (i) the D37 expression vector having an un-evolved TIR and a wild-type nucleotide sequence for the PelB signal peptide upstream of each of the nucleotide sequences for the light and heavy chains of Certolizumab, and (ii) expression vector E83 comprising synthetically engineered TIR for the regulation of the Certolizumab heavy chain expression.

After 20 hours of expression in DASbox® fermenters, Certolizumab purification runs were performed with frozen clarified lysate from expression systems XB102 and XB62. Said clarified lysate was prepared by (1) homogenization for three passages at about 800-900 Bar, (2) centrifugation, and (3) filtering of the supernatant with 0.45 PES (polyethersulfone) filter. More specifically, 9.5 mL of the clarified lysate was loaded onto a CaptureSelect™ CH1-XL column (affinity resin having selectivity for the CH1 domain) coupled to an ÄKTA chromatography system for each purification run and 28 mL of the elution phase, which contained the majority of the assembled Certolizumab, was collected from each run.

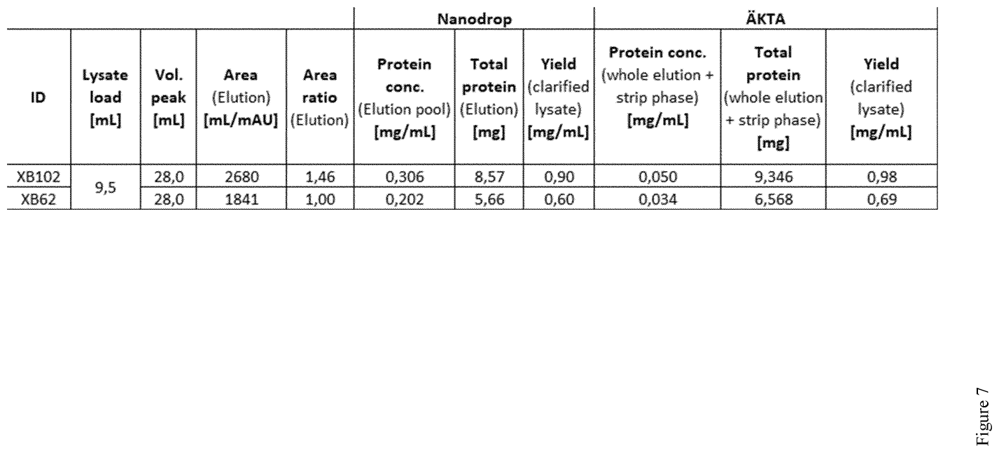

The protein concentration in the collected elution pool was measured by using a NanoDrop® microvolume UV-VIS spectrophotometer at 280 nm wavelength with 1.6 set as the extinction coefficient (see FIG. 7, column titled “Nanodrop” for the results). The total amount of protein in the elution pool could then be calculated by multiplying 28 mL elution with the concentration measured. The total amount of protein in the collected elution pool was then divided by the volume sample loaded onto the column to calculate the yield target protein in the clarified lysate, which was compared between the two batches.

In addition, the two runs were also evaluated by calculating the amount of protein throughout the whole elution phase and strip phase by using Unicorn internal evaluation software (of the ÄKTA system) with 1.6 set as the extinction coefficient (see FIG. 7, column titled “ÄKTA” for the results). The titer value is ⅔ of the yield value, due to volume of cell mass in the harvest which is lost during clarification prior the purification on column.

The results from both the NanoDrop® and ÄKTA analyses summarized in FIG. 7 clearly show that the expression system XB102 which comprises the expression vector E83 containing the synthetically engineered TIR (for the regulation of the Certolizumab heavy chain expression) results in highest yields and titer of Certolizumab (when compared to expression vector D37 which lacks the synthetically engineered TIR for the regulation of the Certolizumab heavy chain expression).

### Example 9—Comparison of Certolizumab Expression in Host Cells XB17 and XB166

Certolizumab expression levels were analyzed when using the E83 expression vector (which comprises synthetically engineered TIR for the regulation of the Certolizumab heavy chain expression) in the following host cells:
XB17 (E. coli W3110 rhaBfs), and
XB166 (E. coli W3110 rhaBfs ΔDegP Δprc sprW148R).

After 20 hours of induced expression in the DASbox® fermenter, 1 ml sample was centrifuged at 13500 rpm for 20 min at 4° C. Pellet and media fraction were separated and the pellet was resuspended with 0.5 ml of 100 mM Tris HCl/10 mM EDTA, pH 7.4. Next, the resuspension was vortexed thoroughly before incubated at 60° C. for 16 hours. The pellet samples were then clarified by centrifugation at 13500 rpm for 20 minutes at 4° C. before the supernatant (extracted periplasmic sample) was collected and treated with DNase (final concentration of 0.02 mg/ml). Samples were filtered with low protein binding syringe filters (0.2 μm, Spartan 13, GE Healthcare). After periplasmic extraction, samples were centrifuged at 14000×g for 5 minutes after which 20 μl supernatant was directly analyzed using affinity column (CH1-XL)-HPLC. Protein concentrations were compared to a standard curve using purified Certolizumab with known concentrations.

The results illustrated in FIG. 8 show that the use of the XB166 host cell results in higher relative yields of Certolizumab when compared with the expression in host cell XB17.

### Example 10—Comparison of Certolizumab Expression in Host Cell XB166 by Using E83 and E111 Expression Vectors

Due to the above described advantageous effects of the E83 expression vector (see Examples 6-9), the E83 expression vector was used as a template to synthetically evolve the light chain TIR (according to the general method described in Example 5). The resulting vector with best technical comprised the synthetically evolved the TIR upstream of the light chain having SEQ ID 20 (TIR-LC). Said vector is in the present invention referred to as E111.

In other words, the E111 expression vector comprises:
a synthetically evolved TIR of SEQ ID 20 (TIR-LC) for the regulation of the expression of the light chain of Certolizumab; and
a synthetically engineered TIR of SEQ ID 21 (TIR-HC) for the regulation of the expression of the heavy chain of Certolizumab

This example is similar to Example 9 but differs in that the difference of Certolizumab expression was instead compared between:
XB166 host cell comprising the E83 expression vector (having the synthetically engineered TIR of SEQ ID NO 21 for the regulation of only the heavy chain of Certolizumab) and wherein the combination of host cell and vector is referred to as XB174 in FIG. 9; and
XB166 host cell comprising the expression vector E111 having the synthetically constructed TIRs of SEQ ID 20 and SEQ ID NO 21 for the regulation of the expression of light and heavy chains of Certolizumab, respectively.

The results illustrated in FIG. 9 show that the use of the expression vector E111 results in higher relative yields of Certolizumab when compared with E83. In other words, the E111 expression vector which comprises:
a synthetically engineered TIR for the regulation of the heavy chain; and
synthetically evolved TIR for the regulation of the light chain, results in higher yields of Certolizumab when compared to E83 which does not have synthetically evolved TIR for the regulation of the light chain expression.

### REFERENCES

1. Holcroft C C et al. Interdependence of activation at rhaSR by cyclic AMP receptor protein, the RNA polymerase alpha subunit C-terminal domain, and rhaR. J Bacteriol. 2000; 182 (23): 6774-6782
2. Hjelm, A. et al. Tailoring Escherichia coli for the 1-Rhamnose PBAD Promoter-Based Production of Membrane and Secretory Proteins. ACS synthetic biology 2017; 6 6:985-994.
3. Wilms, B. et al. High-cell-density fermentation for production of L-N-carbamoylase using an expression system based on the Escherichia coli rhaBAD promoter. Biotechnol. Bioeng. 2001; 73:95-103
4. Kumar, D. et al. QbD Based Media Development for the Production of Fab Fragments in E. coli. Bioengineering 2019; 6, 29.
5. Giacalone M J et al. “Toxic protein expression in Escherichia coli using a rhamnose-based tightly regulated and tunable promoter system. Biotechniques. 2006; 40 (3): 355-364.
6. Choi, J. H. et al. Secretory and extracellular production of recombinant proteins using Escherichia coli. Appl Microbiol Biotechnol 2004; 64, 625-635
7. Wilms B, Hauck A, Reuss M, Syldatk C, Mattes R, Siemann M, Altenbuchner J. High-cell-density fermentation for production of L-N-carbamoylase using an expression system based on the Escherichia coli rhaBAD promoter. Biotechnol Bioeng. 2001 Apr. 20; 73 (2): 95-103.
8. Chen C, Snedecor B, Nishihara J C, Joly J C, McFarland N, Andersen D C, Battersby J E, Champion K M. High-level accumulation of a recombinant antibody fragment in the periplasm of Escherichia coli requires a triple-mutant (degP prc spr) host strain. Biotechnol Bioeng. 2004 Mar. 5; 85 (5): 463-74.
9. Hamilton C M, Aldea M, Washburn B K, Babitzke P, Kushner S R. New method for generating deletions and gene replacements in Escherichia coli. Journal of Bacteriology. 1989; 171 (9): 4617-4622.
10. Link A J, Phillips D, Church G M. Methods for generating precise deletions and insertions in the genome of wild-type Escherichia coli: application to open reading frame characterization. J Bacteriol. 1997 October; 179 (20): 6228-37
11. Mirzadeh K, Martínez V, Toddo S, Guntur S, Herrgård MJ, Elofsson A, Nørholm MH, Daley D O. Enhanced Protein Production in Escherichia coli by Optimization of Cloning Scars at the Vector-Coding Sequence Junction. ACS Synth Biol. 2015 Sep. 18; 4 (9): 959-65

1. A DNA construct for expressing a recombinant protein, wherein the DNA construct comprises:
at least one of the nucleotide sequences of SEQ ID No 20 and 21, wherein a nucleotide sequence of SEQ ID 20 and 21 is a TIR sequence; and
a nucleotide sequence which encodes a signal peptide; and
wherein a nucleotide sequence of SEQ ID No 20 and 21 comprises at least the first 9 nucleotides of said signal peptide encoding sequence.

2. The DNA construct according to claim 1, wherein the TIR sequence transcribes into an RNA motif that functions as the protein translation initiation site in an mRNA transcript.

3. The DNA construct according to claim 1 or 2, wherein the nucleotide sequence which encodes a signal peptide comprises a nucleotide sequence of SEQ ID No 18 when DNA construct comprises a TIR of nucleotide sequence of SEQ ID No 20.

4. The DNA construct according to any one of claims 1-3, wherein the nucleotide sequence which encodes a signal peptide comprises a nucleotide sequence of SEQ ID No 19 when the DNA construct comprises a TIR of nucleotide sequence of SEQ ID No 21.

5. The DNA construct according to any one of claims 1-4, wherein the DNA construct comprises nucleotide sequences of SEQ ID No 20 and SEQ ID No 21, wherein the DNA construct comprises two nucleotide sequences which encode signal peptides, wherein the first nucleotide sequence which encodes a signal peptide comprises a nucleotide sequence of SEQ ID No 18, wherein the second nucleotide sequence which encodes a signal peptide comprises a nucleotide sequence of SEQ ID No 19, wherein the nucleotide sequence of SEQ ID No 20 comprises at least the first 9 nucleotides of the nucleotide sequence of SEQ ID No 18, and wherein the nucleotide sequence of SEQ ID No 21 comprises at least the first 9 nucleotides of the nucleotide sequence of SEQ ID No 19.

6. The DNA construct according to any one of claims 1-5, wherein the DNA construct comprises a nucleotide sequence which encodes said recombinant protein, wherein said recombinant protein is preferably an antibody, wherein said recombinant protein is more preferably a monoclonal antibody, a polyclonal antibody, a chimeric antibody or a fragment of any of said antibodies, wherein said recombinant protein is most preferably certolizumab or certolizumab biosimilar.

7. The DNA construct according to any one of claims 1-6, wherein the nucleotide sequence which encodes a signal peptide is operably linked to the nucleotide sequence which encodes the recombinant protein.

8. The DNA construct according to any one of claims 1-7, wherein the nucleotide sequence which encodes said recombinant protein comprises:
a first nucleic acid sequence which encodes a light chain of an antibody; and
a second nucleic acid sequence which encodes a heavy chain of an antibody.

9. The DNA construct according to claim 8, wherein the nucleotide sequence which encodes a signal peptide is operably linked to:
the first nucleotide sequence which encodes the light chain of an antibody; and/or
the second nucleotide sequence which encodes the heavy chain of an antibody.

10. The DNA construct according to any one of claims 1-9, wherein the DNA construct comprises a Shine-Dalgarno sequence, preferably said Shine-Dalgarno sequence is located upstream from the ATG start codon of the nucleotide sequence which encodes a signal peptide, more preferably said Shine-Dalgarno sequence is located upstream from the ATG start codon of the nucleotide sequence which encodes a signal peptide which is operably linked to the light chain of an antibody.

11. The DNA construct according to any one of claims 8-10, wherein the first nucleotide sequence which encodes the light chain of an antibody encodes an amino acid sequence of SEQ ID No 3.

12. The DNA construct according to any one of claims 8-11, wherein the second nucleotide sequence which encodes the heavy chain of an antibody encodes an amino acid sequence of SEQ ID No 4.

13. The DNA construct according to any one of claims 8-12, wherein the first and second nucleotide sequences which encode for the light and heavy chains of an antibody, respectively, encode amino acid sequences of SEQ ID No 3 and SEQ ID No 4, respectively.

14. The DNA construct according to any one of claims 8-13, wherein the first nucleotide sequence which encodes the light chain of an antibody comprises a nucleotide sequence of SEQ ID No 5.

15. The DNA construct according to any one of claims 8-14, wherein the second nucleotide sequence which encodes for the heavy chain of an antibody comprises a nucleotide sequence of SEQ ID No 6.

16. The DNA construct according to any one of claims 8-15, wherein the first and second nucleotide sequences which encode the light and heavy chains of an antibody, respectively, comprise nucleotide sequences of SEQ ID No 5 and SEQ ID No 6, respectively.

17. An expression vector comprising the DNA construct according to any one of claims 1-16.

18. A host cell comprising the DNA construct according to any one of claims 1-16 or comprising an expression vector according to claim 17, wherein said host cell is a bacterial cell, more preferably Escherichia coli (E. coli), most preferably E. coli comprising a chromosome which comprises a mutation or modification which disables rhamnose metabolism.

19. The host cell according to claim 18, wherein said host cell comprises either (i) a chromosome which comprises a mutation in the nucleotide sequence of the rhaB gene which renders RhaB inactive, or (ii) a chromosome in which the nucleotide sequence encoding RhaB is deleted.

20. The host cell according to claim 18 or 19, wherein said host cell is an E. coli W3110, preferably comprising a chromosome which comprises a frame shift-mutation in the nucleotide sequence encoding RhaB.

21. The host cell according to any one of the claims 18-20, wherein said host cell comprises a chromosome comprising:
a. a frame shift-mutation in the nucleotide sequence encoding RhaB;
b. a degP deletion;
c. a prc deletion; and
d. a sprW148R mutation.

22. The host cell according to any one of claims 18-21, wherein said host cell is E. coli W3110 rhaBfs ΔDegP Δprc sprW148R.

23. An RNA expressed by the DNA construct according to any one of claims 1-16.

24. A method of expressing a recombinant protein comprising the use of the host cell according to any one of claims 18-22.

25. The method according to claim 24, further comprising the step of recovering the recombinant protein from the host cell; optionally further comprising one or more steps of purifying the recovered recombinant protein, preferably by one or more chromatography steps.

26. A recombinant protein obtainable by a method according to claim 24 or 25.

27.-100. (canceled)
